(19) United States
(12) Patent Application Publication
(10) Pub. No.: US 2009/0232762 A1
(43) Pub. Date: Sep. 17, 2009

(54) COMPOSITIONS FOR DELIVERY OF THERAPEUTIC AGENTS

(76) Inventors: May Pang Xiong, Pasadena, CA (US); Glen S. Kwon, Waunakee, WI (US); Younsoo Bae, Madison, WI (US); Kazunori Kataoka, Tokyo (JP)

Correspondence Address: SCHWEGMAN, LUNDBERG & WOESSNER, P.A., P.O. BOX 2938, MINNEAPOLIS, MN, 55402, US

(21) Appl. No.: 12/046,287

(22) Filed: Mar. 11, 2008

(51) Int. Cl. C12N 15/88; A61K 31/7088; C08G 63/00; A61K 47/34

(52) U.S. Cl. 424/78.3; 528/271; 525/540; 514/772.3; 435/440; 514/44 A

(57) ABSTRACT

The invention provides a polyamide compounds and compositions, in addition to methods for their preparation and methods for their use. The invention also provides micelle compositions that include encapsulated therapeutic agents, for example, nucleic acids or prodrugs, and methods for their preparation. The invention further provides methods of delivering therapeutic agents to cells and methods of activating therapeutic prodrugs.

### RELATED APPLICATIONS

This application is a U.S. National Stage Filing under 35 U.S.C. § 111(a) of International Application No. PCT/US2007/024966, filed Dec. 6, 2007, which claims priority under 35 U.S.C. § 119(e) to U.S. Provisional Patent Application No. 60/873,169, filed Dec. 6, 2006, which applications are incorporated herein by reference.

### GOVERNMENT SUPPORT

This invention was made with government support from the National Institutes of Health under NIH Grant R01 AI-43346-08. The United States Government has certain rights in this invention.

### BACKGROUND OF THE INVENTION

Gene Directed Enzyme Prodrug Therapy (GDEPT) is a strategy for minimizing normal tissue toxicities associated with systemic administration of anticancer drugs. GDEPT involves expressing a gene coding for a prodrug-activating enzyme in tumorgenic cells. In typical GDEPT, delivery vectors extravasate into targeted cells, for example, by transport through leaky tumor blood vessels into a cancer tumor. A non-toxic prodrug is then injected intravenously, such that only cells expressing the enzyme convert the prodrugs into toxic agents, thereby minimizing negative side effects to healthy tissues. This therapy has great potential but has yet to be fully developed. Improved gene delivery compositions and methods would aid in the further development of GDEPT.

Viral gene delivery methods, which usually involve transfecting with lentivirus vectors containing a gene of interest are viewed with reservation because of potential problems with immunogenicity and other safety risks. There has been a considerable amount of research in the area of non-viral gene delivery methods, however, current methods still suffer from a variety of drawbacks, including inadequate delivery of vectors and unfavorable toxicity.

Accordingly, there is a need for novel compounds, compositions, and methods for mediating non-viral transfection. There is also a need for novel compositions, such as non-viral vehicles, and methods for mediating gene and prodrug delivery to cells. There is a further need for compositions that are stable, have low toxicity toward normal tissue and function, and can provide for release of therapeutic agents in target tissues or cells.

### SUMMARY

The invention provides a polyamide block polymer including a first block and a second block; wherein the first block includes amino acid units with side chains that can have neutral or negative charges; the second block includes amino acid units with side chains that can have positive charges; wherein at least one side chain of an amino acid of the first block is covalently linked to a poly(ethylene glycol) chain through a hydrazide moiety, and wherein the hydrazide moiety is linked to the poly(ethylene glycol) chain at the N′ nitrogen of the hydrazide through a hydrazone's N═C bond.

The first block can include aspartic acid or glutamic acid units. The second block can include lysine or arginine units. The aspartic acid or glutamic acid units can be covalently linked to poly(ethylene glycol) chains. The molecular weight of the poly(ethylene glycol) chains can be about 500 to about 50,000. A plurality of aspartic acid or glutamic acid units can be covalently linked to poly(ethylene glycol) chains and the poly(ethylene glycol) chains terminate in methoxy, amino, or acetamide groups. The ratio of aspartic acid or glutamic acid units to polyethylene(glycol) chains can be about 1:1 to about 20:1. The polymer can include poly(lysine) units wherein one or more of the lysine side chain amino group is protected with nitrogen protecting groups, such as trifluoroacetate groups or carbonyl-benzyloxy (CBz) groups.

In one embodiment, the first block is poly(aspartic acid) comprising about 20 to about 60 aspartic acid units. In certain embodiments, the second block is poly(lysine) comprising about 30 to about 80 lysine units. In some embodiment, the first block is poly(glutamic acid) comprising about 20 to about 60 arginine units. In certain embodiments, the second block is poly(arginine) comprising about 30 to about 80 arginine units.

Throughout this descriptions, it is important to note that the various embodiments of the invention include replacing one or more units of a ‘lysine’ block with arginine units, aspartate units, aspartate unit derivatives such as aspartate with a diethyl triamine side chain (an N-(2-aminoethyl)-2-aminoethyl group in the side chain), or an imidazole acetic acid group on a side chain. Additionally, embodiments also include replacing any aspartic acid unit with a glutamic acid unit, optionally in combination with a replacement of one or more lysine units. In some embodiments, a typical polymer of the invention will include at least about 60 repeating units. In other embodiments, the polymers of the invention may include up to about 150 repeating units.

The invention also provides a polyamide block polymer comprising at least one block of poly(aspartic acid) units and at least one block of poly(lysine) units; wherein at least one side chain of an aspartic acid unit is covalently linked to a poly(ethylene glycol) chain through a hydrazide moiety, and wherein the hydrazide moiety is linked to the poly(ethylene glycol) chain at the N′ nitrogen of the hydrazide through a hydrazone bond.

A plurality of aspartic acid units can be covalently linked to poly(ethylene glycol) chains and the molecular weight of the poly(ethylene glycol) chains can be about 500 to about 10,000. A plurality of aspartic acid units can be covalently linked to poly(ethylene glycol) chains and the poly(ethylene glycol) chains can terminate in various organic groups, such as methoxy, amino, or acetamide groups, or combinations thereof. The ratio of aspartic acid units to polyethylene(glycol) chains can be about 1:1 to about 20:1, or any ratio in between. The polymer can include poly(lysine) units wherein the side chain amino group is protected with a nitrogen protecting group, for example, a trifluoroacetate group or a carbonylbenzyloxy group. Protecting groups can optionally be removed before the polymer is used to prepare a micelle that incorporates various cargo.

The poly(aspartic acid) block can include about 20 to about 60 aspartic acid units. The poly(lysine) block can include about 30 to about 80 lysine units.

The invention further provides a micelle composition that includes a polymer as described herein and a therapeutic agent, wherein the polymer substantially encapsulates the therapeutic agent and the poly(ethylene glycol) chains of the polymer align toward the outside surface of the micelle. The therapeutic agent can include a nucleic acid, a gene, a drug, a prodrug, or a combination thereof. The nucleic acid can be plasmid DNA encoding gene products or RNAi.

Also provided is a polyamide polymer comprising amino acid units with side chains that can have positive, neutral, or negative charges; wherein at least one side chain of an amino acid of the polyamide is covalently linked to a poly(ethylene glycol) chain through a hydrazide moiety, and wherein the hydrazide moiety is linked to the poly(ethylene glycol) chain at the N′ nitrogen of the hydrazide through a hydrazone bond.

The amino acid units can include a variety of amino acids, including aspartic acid units, lysine units, or both. A plurality of aspartic acid units can be covalently linked to poly(ethylene glycol) chains. The molecular weight of the poly(ethylene glycol) chains can be about 500 to about 10,000. A plurality of aspartic acid units can be covalently linked to poly(ethylene glycol) chains and the poly(ethylene glycol) chains can terminate in PEG-capping groups, for example, amino or acetamide groups. The ratio of aspartic acid units to polyethylene(glycol) chains can be about 1:1 to about 20:1, or any ratio in between. One or more of the lysine side chain amino group can be protected with a nitrogen protecting group, such as trifluoroacetate groups or carbonylbenzyloxy groups.

The polymer can include about 5 to about 100 amino acid units in the first block. The polymer can include about 10 to about 150 amino acid units in the second block. In one embodiment, the amino acids can be aspartic acid or glutamic acid in the first block, and lysine or arginine in the second block. The polymer can also include about 5 to about 60 units, about 20 to about 100 units, about 10 to about 80 units, or about 20 to about 60 units, in the first block. The polymer can also include about 10 to about 80 units, about 20 to about 150 units, about 20 to about 120 units, about 30 to about 100 units, or about 30 to about 80 units, in the second block.

The invention further provides a polymer comprising formula I:

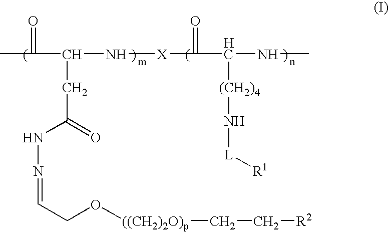

wherein m is about 20 to about 60; n is 0, or about 30 to about 80; p is selected such that the molecular weight of the poly(ethylene glycol) chain is about 500 to about 20,000. The group L can be a direct bond from NH to R¹, or L can be a carbonyl or sulfonyl group. R¹ can be H, or together with the carbonyl to which it is attached, can be a nitrogen protecting group, for example, trifluoroacetate or carbonylbenzyloxy. R² can be hydrogen, hydroxy, (C₁-C₁₂)alkyl, alkoxy, —NHR³ wherein R³ is hydrogen, an amino protecting group, or any PEG-capping group; and X can be a direct bond, an organic linking group, or a group of about 1 to about 100 amino acids; or a salt thereof.

In certain embodiments, m can be about 30 to about 50, n can be about 40 to about 60, and p can be selected such that the molecular weight of the poly(ethylene glycol) chain is about 2,000 to about 10,000. In various embodiments, R² can be —NHR³ wherein R³ is an acetyl group.

The invention also provides a polymer comprising formula II:

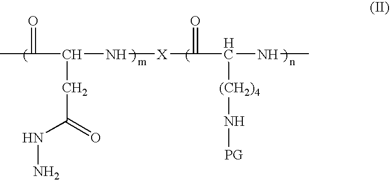

wherein m is about 20 to about 60; n is 0, or about 30 to about 80; each PG is independently hydrogen or an amino protecting group; and X is a direct bond, an organic linking group, or a group of about 1 to about 100 amino acids; or a salt thereof.

Also provided is a method of activating a therapeutic prodrug that includes contacting a micelle composition described herein and a cell, wherein the micelle composition contains a nucleic acid, gene, or vector that encodes an enzyme that converts the prodrug to an active drug. The method can further include introducing the prodrug to the vicinity of the cell after the cell has synthesized the enzyme so that the enzyme activates the prodrug to the active drug. The introduction of the prodrug to the vicinity of the cell can be via systemic delivery, such as by IV or other injection. The formulation for injection can include a pharmaceutically acceptable carrier or diluent, for example, saline that optionally includes a buffer.

Further provided is a method of delivering a nucleic acid to a cell that includes encapsulating nucleic acid in a micelle comprising a polymer described herein; and contacting a cell with the micelle. The nucleic acid can be a gene, a vector, or a specific type of RNA, such as siRNA, or a plasmid that codes for RNAi.

The invention additionally provides a method of treatment for a disease or a condition in a human or an animal by administering a micelle composition that includes a polymer disclosed herein and a therapeutic agent, such as a nucleic acid or a drug. The nucleic acid can encode for a therapeutic protein, such as an enzyme. A prodrug can then be administered to a patient, whereby cells that produce the enzyme then activate the prodrug to a drug, and then the drug takes its effect on the cell.

One aspect of the invention involves the preparation of a pH-sensitive polyamide copolymer, such as a poly(Aspartate-Hydrazide-PEG)-b-poly(L-Lysine) block-copolymer. The poly(L-Lysine) segment of the polyamide can be used to condense an agent within an association of polyamide molecules, such as in a micelle core or polyamide particle core. The agent can be a therapeutic agent, such as a drug or a gene therapy agent, for example, a pDNA, or the agent can be a diagnostic agent or an imaging agent. Aldehyde-PEG groups can be attached, for example, through the formation of hydrazone bonds to hydrazide groups, forming a variable brush layer in the particle shell, composed of acid labile PEG moieties. At a pH of less than approximately 7.0, these acid labile PEG moieties can be released from the polymer, leaving behind hydrazide groups that can enhance the escape of pDNA from endolysosomes via membrane disruptions. The rapid release of the PEG brush layer can further enhance agent unpacking, for example, pDNA unpackaging.

The compositions and methods thus provide non-viral transfection vehicles with reduced toxicity compared to standard poly(lysine) delivery vehicles. The compositions and methods also improved in vivo stability, wherein the rate of release of encapsulated agents can be tuned by varying the amount of PEG groups on the cationic polymers.

The invention thus provides novel polymers, polymer compositions, including micelles, and methods of making and using the polymers and polymer compositions. The polymers and compositions can be used to treat diseases or disorders of mammals, for example, using GDEPT. The polymers and compositions can also be used to prepare a medicament to treat diseases in mammals, for example, cancer in a human. Also provided are useful intermediates for the preparation of the polymers disclosed herein.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention, however, one skilled in the art will understand that portions of the example or aspect may be used in combination with other examples or aspects of the invention.

FIG. 1 illustrates a block-copolymer polyplex according to an embodiment of the invention. The first structure illustrates hydrazine groups exposed at the surface of the polyplex particle. The second structure illustrates post-PEGylation of the polyplexes, which include a brush layer of PEG. The second arrow indicates pH-triggered release and degradation of the cationic polymer in a cell's reductive environment.

FIG. 2(a) illustrates a GPC (10 mM LiCl in DMF) of p(Asp-Hyd)₃₆-PLL(TFA)₅₃ reaction with aldehyde-PEG7k at a 1:1 molar ratio after 2 and 5 days incubation at RT. Peak a is un-reacted PEG7k, peak b is un-reacted p(Asp-Hyd)₃₆-PLL(TFA)₅₃ block-copolymer and peak c is the multimodal distribution of pre-PEGylated block-copolymers.

FIG. 2(b) illustrates a GPC (10 mM LiCl in DMF) of p(Asp-Hyd)₃₆-PLL(TFA)₅₀ reaction with aldehyde-PEG7k at a 1:0.5 molar ratio after 2 and 5 days incubation at RT. Peak a is un-reacted PEG7k, peak b is un-reacted p(Asp-Hyd)₃₆-PLL(TFA)₅₃ block-copolymer and peak c is the unimodal distribution of pre-PEGylated block-copolymers.

FIG. 2(c) illustrates a GPC (10 mM LiCl in DMF) of p(Asp-Hyd)₃₆-PLL(TFA)₅₀ reaction with aldehyde-PEG7k at a 1:0.1 molar ratio after 2 and 5 days incubation at RT. Peak a is un-reacted PEG7k, peak b is un-reacted p(Asp-Hyd)₃₆-PLL(TFA)₅₃ block-copolymer and peak c is perhaps pre-PEGylated block-copolymers.

FIG. 3 illustrates transfection efficiency of PEI25, BC, and PBC at N/P 70-100 in MDA-MB-231 cells.

FIG. 4 illustrates the protonation profile of polyethyleneimine PEI-25 (MW=25,000 g/mol), a block copolymer according to an embodiment of the invention (“BC”), and poly(l-lysine) (DP 44) (“PLL”), as a function of pH, conducted according to and embodiment of Example 2.

FIG. 5 illustrates the acid sensitivity of p(Asp-Hyd-PEG)-poly(L-lysine(TFA)), according to an embodiment of the invention. Release of PEG from the block-copolymer was verified by GPC analysis by the addition of various amounts of TFA and incubating overnight. At a pH of 7.4, very little PEG was released. At a pH<7.0, the release of PEG was immediate and pH-dependent, with over 50% PEG release within 20 minutes of incubation at pH 3.0.

FIG. 6 illustrates a SEC example of a block copolymer aggregation after PEG following 20 minutes incubation at pH 3, 5.5, and 7.4, according to an embodiment.

FIG. 7 illustrates (A) successful ligation of the yCD (˜500 bp) into the Hermes vector and screening of bacterial colonies; and (B) propagation of the vector and digest to verify gene inserted into the vector; lane 1=DNA ladder, lane 2=undigested Hermes-yCD, lane 3=digested HermesyCD, lane 4=lane 5=RtAb(−) vector, according to an embodiment of Example 2.

FIG. 8 illustrates (A) expression of yCD as shown by (A): a Coomassie-stained gel of the crude lysate after induction of yCD; and (B) a Western blot of crude protein extracts probed with Tetra-His conjugate.

FIG. 9 illustrates transfection activity at increasing equivalent N/P ratios and toxicity profiles of the free polymers in serum media for (A) C2C12; and (B) MDA-MB 231.

FIG. 10 illustrates a Gene Directed Enzyme Prodrug Therapy (GDEPT) concept according to an embodiment of the invention.

FIG. 11 illustrates a pH-sensitive poly(Aspartate-Hydrazide-PEG)-b-poly(L-Lysine) block-copolymer particle and its release of PEG groups, according to an embodiment of the invention.

FIG. 12 illustrates the formation of polyplex micelles through the electrostatic interaction between block catiomers and plasmid DNA, according to an embodiment of the invention.

FIG. 13 illustrates structures of the block copolymers:

a) poly(aspartate-hydrazide)-block-poly(L-lysine) (BC); and b) pH-sensitive poly(aspartate-hydrazide-PEG)-block-poly(L-lysine) (pH-PBC), and covalent poly(aspartate-hydrazide-PEG)-block-poly(L-lysine) (cov-PBC).

FIG. 14 illustrates a) cationic polyplexes formed with PLL (or PEI) are cytotoxic to cells; in contrast, neutral shielded particles [parts b), c), and d)] cause minimal membrane damage during endocytosis: following a decrease in pH, hydrazide amino groups protonate b) and/or acid-labile PEG chains are released c); this increases endosomal membrane disturbances with the resulting charged particle and enhances escape into the cytosol (pH 7.4), whereupon sensing the new pH, hydrazide amino groups deprotonate again, once more imparting neutral properties to the PIC and minimizing intracellular toxicity; d) cov-PBC particles cannot release PEG chains following a decrease in pH, and this can minimize particle interactions with the endosomal membrane.

FIG. 15 illustrates protonation profiles comparing branched PEI, BC, and PLL. As expected, PEI can buffer over a relatively wide pH range. BC carries little buffering capacity, similar to PLL (note: error bars omitted to simplify profile).

FIG. 16 illustrates isoelectric points (pI) of the polymer in complex with 1 μg pGL3; values indicate equivalent N/P ratios: a) PEI (25,000 g mole⁻¹), pI (w/w)=0.20 μg, N/P=1.1; b) BC (poly(Asp-Hyd)₃₆-PLL₄₉) (11,000 g mol⁻¹), pI (w/w)=0.65 μg, N/P=0.9; c) PLL₄₄ (9,200 g mol⁻¹), pI=0.40 μg, N/P=0.7; d) pH-PBC (five PEG chains per BC, 46000 g mol⁻¹), pI=2.5 μg, N/P=0.96; e) cov-PBC (six PEG chains per BC, 53,000 g mol⁻¹), pI=4 μg, N/P=1.0. Electrostatic interactions occur mostly between lysine groups and pGL3, as BC and PBC polymers have similar N/P ratios of ˜1.

FIG. 17 illustrates a) size and b) ξ profiles for BC (white) and pH-PBC (gray) at the indicated pH values, according to an embodiment of the invention.

FIG. 18 illustrates PEG release monitored by GPC: pH-PBC was injected into an OHpak SB-806M GPC column (injection volume: 20 μL; polymer: 2 mg mL⁻¹; of HEPES eluent: 10 mm, pH 7.4, 0.75 mL min⁻¹, 30° C.; Shodex, Kawasaki, Japan), and PEG release was detected by refractive index (RI) for 12 hours; a) PEG falls off upon incubation at pH<7.0, but the hydrazone bond is relatively stable at pH 7.4, with less than 10% PEG released over 12 hours; the reversible nature of the hydrazone bond leads to an equilibrium state in the release profiles at different pH's; and b) sample curves at t=2 hours are shown with pH values indicated.

FIG. 19 illustrates membrane toxicity of polymers at concentrations of 20 and 40 μg mL⁻¹ toward MDA-MB-231 (human breast cancer) cells in DMEM serum-free media; polymers were incubated both in free form [parts a) and c)] and also pre-complexed to 1 μg pGL3 [parts b) and d)].

FIG. 20 illustrates metabolic toxicity of polymers incubated for 4 hours at various concentrations toward MDA-MB-231 (human breast cancer) cells in DMEM serum-containing media. Polymers were incubated in both free form and pre-complexed to 1 μg pGL3; a) free polymers with cationic components are toxic to cells at higher concentrations, with PEI being more toxic than BC (*p<0.05 using two-way ANOVA); and b) when complexed, BC results in negligible toxicity to the cell, similar to pH-PBC particles (*p>0.05 using two-way ANOVA).

FIG. 21 illustrates transfection efficiency of the polymers in MDA-MB-231 (human breast cancer), C2C12 (murine myoblast), COS-7 (Green African monkey kidney), MCF-7 (human breast cancer) and 4T-1 (murine colon cancer) cells when incubated in the respective serum-containing media for 4 hours; all polymers were formed at N/P=20 (optimal ratio determined in tests with N/P=7-50) with 2 μg pGL3; pH-PBC and BC PICs consistently effect significantly higher gene expression relative to cov-PBC across all cell lines (*p<0.05 using one-way ANOVA in each case).

### DETAILED DESCRIPTION

The invention provides polymers, particularly block co-polymers, that can have refined properties that make them “tunable” in response to various environmental conditions, addition of ligands, excipients, etc. Block co-polymers that include PEG groups are of interest because PEG is unique in its ability to facilitate transfer of appended agents across cell membranes. PEG is both water soluble and membrane permeable.

The inclusion of multiple PEG residues in the polymers disclosed herein allows for the covalent yet labile attachment of targeting ligands and tenability or modulation of the release of agents, such as DNA packages, into the polycationic core. In particular, the contents of the particle can be released upon hydrolytic removal of PEG residues in response to slightly lower than physiological pH, such as the pH found in cancerous cells.

One embodiment of the invention comprises a poly(PEG)-poly(lysine) copolymer that encapsulates a vector encoding the non-mammalian enzyme yeast cytosine deaminase (yCD), which has shown potential in activating the prodrug 5-fluorocytosine (5FC) into cytotoxic 5-fluorouracil (5FU) in several tumor models. Cytosine deaminase can be obtained both from S. Cerevisiae (yeast) and E. Coli (bacteria). Both the prodrug and the gene encoding the enzyme can be preferentially delivered to tumor cells because the slightly more acidic extracellular environment, combined with the leaky vasculature of tumor cells, favors delivery of the DNA vector and prodrug to tumor cells.

Variations of this embodiment can be considered a platform for the creation of a library of tunable poly(PEG)-polycation copolymers with modulatable gene and therapeutic agent delivery properties. Thus, the invention adds to the compendium of non-viral vehicles for tunable, specific gene and prodrug delivery into cells.

The invention also provides nonviral gene vectors from synthetic catiomers (polyplexes) as alternatives to viral vectors. One embodiment employs the use of poly(ethylene glycol) (“PEG”)-linked block catiomers, which form a nanoscaled core-shell polyplex with biocompatible PEG palisades. A schematic diagram of this approach is illustrated in FIG. 12. The catiomers can spontaneously associate with pDNA to form polyplex micelles at the sub-100-nm scale with a dense and hydrophilic PEG palisade surrounding the polyplex core. These polyplex micelles with PEG palisades can display high colloidal stability under physiological conditions and can afford appreciable levels of reporter-gene expression to various cell lines even after preincubation in a serum-containing medium. Notably, the polyplex micelles can demonstrate longevity in blood circulation, offering the possibility of their use in systemic gene delivery.

A series of PEG-linked block catiomers with various amine functionalities have been prepared by a simple and affordable synthetic procedure based on aminolysis reactions. These PEG-linked block catiomers can be used as gene carriers. The catiomers with various side chains are capable of efficient and low toxicity transfection even toward primary cells, highlighting important structural factors of the cationic units in the construction of polyplex-type gene vectors. Moreover, the polyplex micelle for transfection with cells, for example, primary osteoblasts, allows for therapeutic methods, including bone regeneration in vivo, mediated by nonviral gene transfection.

### DEFINITIONS

References in the specification to “one embodiment”, “an embodiment”, “an example embodiment”, etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

A “block copolymer” refers to a polymer with repeating units of one type adjacent to each other in a linear manner to form a block, which is linked, for example, through a covalent bond to a second block made up of repeating units of a second type, which are adjacent to one another in a linear manner to form a second block of the block copolymer.

The first block of the polyamide block polymer includes amino acid units derived from amino acid units with side chains that can have neutral or negative charges and the second block of the polyamide block polymer includes amino acid units derived from amino acid units with side chains that can have positive charges.

The phrase “amino acid units derived from amino acid units with side chains that can have negative charges” refers to amino acid units (repeat segments of a polyamide) whose side chains possess a group that can form an anion, for example, at a slightly basic pH, such as under mammalian physiological conditions. In some embodiments, the group can form an anion at intracellular pH. The group can also be uncharged at a neutral pH. Examples of suitable amino acids include aspartic acid, glutamic acid, and derivatives thereof.

The amino acid units of the first block can undergo a variety of reactions to provide multifunctional polyamides. Some side chain groups may remain as the initial unreacted side chains. Others can be converted to hydrazide groups. In some embodiments, more than 90% of the first block side chains will be converted to hydrazide groups. Many of these groups can be further reacted to provide hydrazone groups that link the amino acid unit to PEG groups, for example, by a condensation reaction between the N′ nitrogen and an aldehyde group of a PEG chain.

The phrase “amino acid units derived from amino acid units with side chains that can have positive charges” refers to amino acid units (repeat segments of a polyamide) whose side chains possess a group that can form a cation, for example, at a slightly acidic pH, such as under certain mammalian physiological conditions. In some embodiments, the group can form a cation at certain intracellular pH ranges, such as at a pH less than 7. The group can also be uncharged at a neutral pH. Examples of suitable amino acids include lysine, arginine, glutamine, histadine, and derivatives thereof. One suitable derivative includes an imidazole-acetic acid-lysine group such as described by Langer and coworkers (PNAS 2001, 98(3), 1200-1205).

The term “hydrazone bond” refers to the imine (N═C) bond of a hydrazone. In other words, the hydrazone bond refers to the bond formed between the N′ nitrogen of a hydrazine or hydrazide, and a carbonyl group of an aldehyde or ketone, which results in the formation of an N═C bond. The term hydrazone refers to an N-alkylidene derivative of a hydrocarbyl derivative of a hydrazine. The term hydrazide refers to a hydrazine that has an acyl substituent.

The term “therapeutic agent” refers to biologically active agents, prodrugs, or drugs, including, for example, any organic or inorganic small molecule compound (e.g., a molecule with a molecular weight of less than about 700, or less and about 500), polymeric species (including nucleic acids (DNA, such as pDNA, and RNA), proteins, peptides, hormones, carbohydrates, and derivatives thereof), lipids and mixtures thereof, wherein said drug or agent can be administered in vivo (in humans or animals) for the treatment of a disease, condition, or disorder. In one embodiment, the micelle cargo can be lactate dehydrogenase (LKD). In other embodiments, the micelle cargo can be a plasmid that encodes for an enzyme that can activate a prodrug to a drug.

When the therapeutic agent is pDNA, it can either be used for expression of a prodrug activating protein, or the pDNA can include sequences that allow for transposition of a gene into a host genome. Such a method can be used for long term protein expression of, for example, a deficient gene, for treating a genetic disorder. One significant advantage of non-viral vehicles over viral vehicles is that the micelle polyplexes described herein can encapsulate very large sized agents, such as a plasmid with about 5K or about 6K base pairs. For example, pGL3 plasmid (5280 bp) (Promega Corp.) was used as a reporter assay in various experiments. One can increase the amount of polymer in proportion to size of pDNA, using the suitable N/P ratio, for example, a N/P ratio of about 20, for many of the polymers disclosed herein. The N/P 1 for pGL3 was determined by running agarose gels, defining a suitable ratio as the lowest amount of polymer it takes to retard 1 μg of pGL3 (pDNA). This ratio will of course change as other forms of DNA are selected for use in the micelles.

Other therapeutic agents include siRNAs, which are now known to be tolerated and effective in several different tissues. Plasmids encoding RNAi can be used as the therapeutic agent of the micelles of the invention. Certain of these plasmids are available from InvivoGen, San Diego, Calif. RNAi-based drugs currently in pre-clinical development include those targeting respiratory syncytial virus, hepatitis C, HIV, Huntington's disease and several other neurodegenerative disorders (Check (2005) Nat Med 11(3):243-244; Schmidt Knockout Punch. The Promise of RNAi. bio.com, New & Features 2005 Jun. 28). Phase I clinical trials are underway for several siRNA-based drugs targeting age-related macular degeneration (AMD), and Phase II trials have recently started for one siRNA candidate for AMD (www.sirna.com/sirnaproduct/sirna-027.html; www.acuitypharma.com/press.asp). Cellular delivery and stabilization are critical hurdles to overcome in development of viable siRNA-based drugs, therefore the drug delivery vehicles described herein should provide needed help in this important area of therapy. See Soutschek et al. (2004) Therapeutic silencing of an endogenous gene by systemic administration of modified siRNAs. Nature 432(7014):173-178.

A “prodrug” is a pharmacological substance that can be administered in an inactive (or significantly less active) form that the active compound that the substance is metabolized in vivo. Some specific examples of prodrugs include, but are not limited to, enalapril, valaciclovir, levodopa, chloramphenicol, codeine, and gamma-butyrolactone (GBL).

The term “micelle” refers to a supermolecular structure having a core-shell form. Micelle formation is entropy driven and water molecules are typically excluded into the bulk phase. When above critical micelle concentration (CMC), amphiphilic portions of the polymer employed aggregate into structured micelles. Polymeric micelles are typically spherical and can have nanoscopic dimensions in the range of about 1 to about 250 nm, typically in the 20-100 nm range. This is advantageous because circulating particles less than about 200 nm can avoid filtering by interendothelial cell slits at the spleen. Additionally, delivery vehicles of less than about 150 nm are much more efficiently taken up by cells. Polymeric micelles have been shown to circulate in the blood for prolonged periods and capable of targeted delivery of therapeutic agents, for example, nucleic acids or poorly water-soluble compounds. Upon disassociation, micelle unimers are typically <50,000 g/mol, permitting elimination by the kidneys. These properties allow for prolonged circulation with little or no buildup of micelle components in the liver that could lead to storage diseases. A “micelle composition” as used herein is any composition that includes a micelle, for example, a novel micelle as described herein.

The term “PEG” refers to poly(ethylene glycol) and derivatives thereof. The molecular weight of the PEG chain can be about 500 to about 20,000. In certain embodiments, the PEG group can have a molecular weight of about 1,000 to about 20,000, about 2,000 to about 15,000, about 3,500 to about 12,000, or about 3,000 to about 9,000. In other embodiments, the PEG groups can have a molecular weight of about 4,000 or about 7,000. PEG groups can terminate in any variety of groups including hydroxy, alkyl, alkoxy, aryl, arylalkyl, amino, and the like, referred to herein as PEG-capping groups.

The term “PEG-capping group” refers to a group at the end of a PEG chain. The PEG chain can terminate in any variety of groups including hydroxy, alkyl, alkoxy, aryl, arylalkyl, amino, and the like. Other PEG-capping groups that can be used include acyl groups such as acetyl, acryloyl, and benzoyl, various silane protecting groups, benzyl groups, or heterocyclic groups, or the terminal hydroxy group of the PEG can be oxidize to an aldehyde or a carboxylic acid (i.e., the CH₂CH₂—R² group of formula I is oxidized to CH₂CO₂H).

PEG groups can also have an amino acid capping group, such as a group of 1 to about 30 amino acids. In one embodiment, the PEG capping group is melittin, a 26 amino acid membrane disrupting agent. In another embodiment, the melittin can be attached directly to a amino acid unit of the polymer backbone, allowing it to be exposed when the PEG groups of the polymer are hydrolyzed from side chain to which they are linked. Also, the amount of PEG groups relative to the number of amino acid units in a particular block can be effectively adjusted by linking branched PEG groups to the appropriate linking functional groups of the amino acid side chains. It should also be noted that the skilled artisan can control the substitution rate of hydrazide groups on the PEG conjugating block so that carboxyl groups or other functional groups can be introduced to capture small molecules as well as ions. This may induce more effective endosomal escape by affecting ion channels and material transport in the cell.

The term “organic linking group” refers to a group that includes at least one carbon atom that links two other groups together, for example, two polymer blocks. In some embodiments, the organic linking group can be a group of the formula -X¹-X²—C(═Y*)-Z¹-Z²- wherein X¹, X², Y*, Z¹, and Z² are each independently O, S, NH, CH₂, or absent.

The term “protecting group” refers to any group which, when bound to a hydroxyl, nitrogen, or other heteroatom prevents undesired reactions from occurring at this group and which can be removed by conventional chemical or enzymatic steps to reestablish the ‘unprotected’ hydroxyl, nitrogen, or other heteroatom group. The particular removable group employed is often interchangeable with other groups in various synthetic routes. Certain removable protecting groups include conventional substituents such as, for example, allyl, benzyl, acetyl, chloroacetyl, thiobenzyl, benzylidine, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a hydroxyl functionality and later selectively removed either by chemical or enzymatic methods in mild conditions compatible with the nature of the product. These protecting groups can likewise be used to protect a nitrogen atom.

A large number of protecting groups and corresponding chemical cleavage reactions are described in Protective Groups in Organic Synthesis, Theodora W. Greene (John Wiley & Sons, Inc., New York, 1991, ISBN 0-471-62301-6) (“Greene”, which is incorporated herein by reference in its entirety). Included therein are nitrogen protecting groups, for example, amide-forming groups. In particular, see Chapter 1, Protecting Groups: An Overview, pages 1-20, Chapter 2, Hydroxyl Protecting Groups, pages 21-94, Chapter 4, Carboxyl Protecting Groups, pages 118-154, and Chapter 5, Carbonyl Protecting Groups, pages 155-184. See also Kocienski, Philip J.; Protecting Groups (Georg Thieme Verlag Stuttgart, New York, 1994), which is incorporated herein by reference in its entirety. Some specific protecting groups that can be employed in conjunction with the methods of the invention are discussed below.

Typical nitrogen protecting groups described in Greene (pages 14-118) include benzyl ethers, silyl ethers, esters including sulfonic acid esters, carbonates, sulfates, and sulfonates. For example, suitable nitrogen protecting groups include substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxy-phenyldiphenylmethyl, di(p-methoxyphenyl)phenylmethyl, tri(p-methoxy-phenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxy-phenylsilyl); esters (formate, benzoylformate, acetate, choroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), pivaloate, adamantoate, crotonate, 4-methoxycrotonate, benzoate, p-phenylbenzoate, 2,4,6-trimethylbenzoate (mesitoate)); carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, miscellaneous esters (2,6-dichloro-4-methyl-phenoxyacetate, 2,6-dichloro-4-(1,1,3,3 tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate (tigloate), o-(methoxycarbonyl)-benzoate, p-poly-benzoate, α-naphthoate, nitrate, alkyl N,N,N′N′-tetramethyl-phosphorodiamidate, n-phenylcarbamate, borate, 2,4-dinitrophenylsulfenate); and sulfonates (sulfate, methanesulfonate (mesylate), benzenelsulfonate, benzylsulfonate, tosylate, triflate).

Specific ranges, values, and embodiments provided herein are for illustrative purposes and do not otherwise limit the scope of the invention, as defined by the claims. The term “about” is intended to encompass variations in physical values, and in amounts of ingredients and the like, owing to variations in weighing and other measurement techniques, purity of ingredients, and other factors, as would be known to the art worker. Such variations are often no more than about ±0.5%. The term “about” can thus indicate a variation of +5 percent, or ±10 percent of the value specified; for example about 50 percent carries a variation from 45 to 55 percent; or the term can indicate ±1, 2, or 3 integers from the value specified.

### Gene Directed Enzyme Prodrug Therapy (GDEPT)

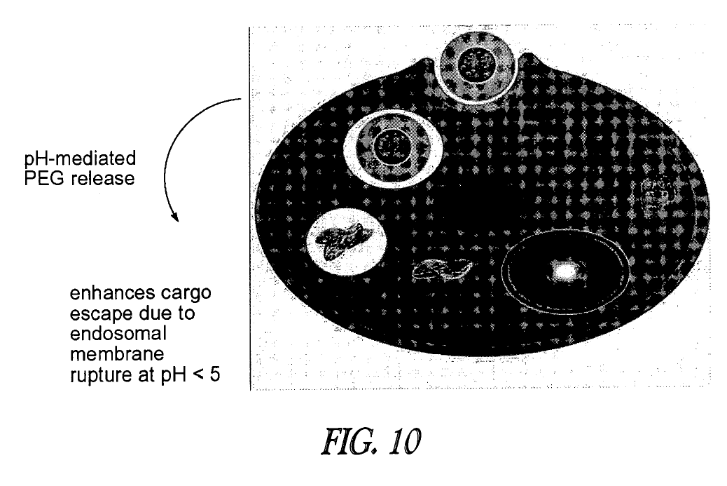

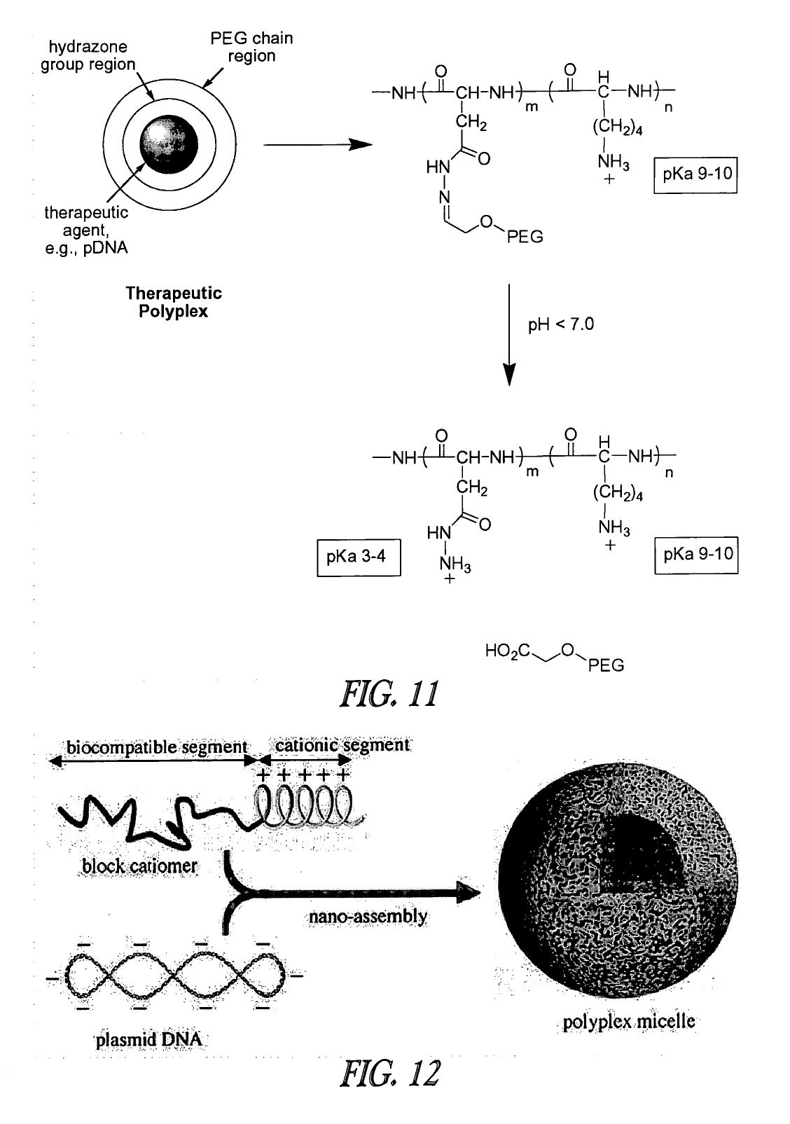

Gene Directed Enzyme Prodrug Therapy (GDEPT) is a strategy for minimizing normal tissue toxicities associated with systemic administration of anticancer drugs. FIG. 10 illustrates a schematic showing this approach. First, a gene coding for a prodrug activating enzyme (e.g. yeast cytosine deaminase) is expressed in tumorgenic cells via a specific delivery vector, for example, a polyplex particle that includes a p(Asp-Hyd-PEG)-poly(L-lysine(TFA)) coating. A pH-triggered release of PEG from p(Asp-Hyd)-b-PLL in the acidic extracellular environment of tumor cells and endolysosomes (6.44<pH<6.79), wherein the polyplex particle displays reduced toxicity due to its PEG coating; inside the cell, pH<5 aids pDNA release from the polyplex particle due to endocytosomal rupture as a result of the hydrazine groups of the polyplex polymer coating after pH-driven PEG release, thus unpackaging the pDNA, and allowing for the pDNA to be incorporated into the nucleus.

The long circulating nanoparticles extravasate across leaky tumor blood vessels into the cancer. New proteins are synthesized by the nucleus that express the incorporated gene coding, e.g., the encoded enzymes. A non-toxic prodrug such as 5-fluorocytosine is injected intravenously. Only cells expressing the enzyme can convert prodrugs into toxic drug molecules (e.g. 5-fluorouracil), thereby minimizing negative side effects to healthy tissues.

A pH-sensitive poly(Aspartate-Hydrazide-PEG)-b-poly(L-Lysine) block-copolymer particle and its release of PEG groups, according to an embodiment of the invention, is illustrated in FIG. 11. Poly(L-Lysine) can be used to condense pDNA in a polyplex core. PEG groups are attached through the formation of hydrazone bonds to hydrazide groups, forming a variable brush layer in the shell. At pH<7.0, acid labile PEG is released from the polymer, leaving behind hydrazide groups that enhance the escape of pDNA from endolysosomes via membrane disruptions. The rapid release of the PEG brush layer may further enhance pDNA unpackaging. The variables m and n can be about 2 to about 200, and in various embodiments, about 20 to about 100, or about 40 to about 60, depending on reaction conditions and the desired properties of the polyplex polymers.

The non-viral delivery vehicles described herein can furthermore deliver pDNA to cancer cells. Upon deliver of the pDNA to the cell interior, the cancer cells can then code for antigens and cytokines to illicit or enhance the body's immune response toward cancer cells. Today nearly 40% of all gene therapy clinical trials have used genes coding for antigens and cytokines (using mostly viral vehicles) with about 70% of trials targeting cancer diseases. Gene therapy agents that can be used in conjunction with the micelles described herein include those being studied in worldwide clinical trials. See for example, http://www.wiley.co.uk/genetherapy/clinical/countries.html. One can also deliver tumor suppressor genes to arrest cancer cell development. Useful information on RNA interference can be obtained from Ambion, Austin, Tex., and from their technical resources and technical notes found at www.ambion.com.

### Polyplex Micelles

Polyplex micelle (or “poly-ion complexes”) can be prepared by various methods, including cosolvent evaporation methods. Micelles can be prepared by mixing solutions of the cationic polymers disclosed herein and pDNA in various ratios of N/P, for which N is the total number of amine groups in the block catiomer and P represents the number of phosphate units in the pDNA. For example, a polymer and an agent can be dissolved in a water miscible solvent system. The solution can be slowly added to a vigorously stirred aqueous solution, followed by solvent evaporation. The resulting composition can be nanofiltered and/or centrifuged to remove unincorporated agent. Other useful techniques for preparing micelles have been reported by Kwon and coworkers, Pharm. Res. 2004, 21, 1184-1191.

The ratio of the solvent phase to the aqueous phase can be adjusted to adjust the size of the resulting micelles. For example, in one embodiment, using a lower proportion of the solvent phase results in smaller micelles. Suitably the ratio of solvent phase to aqueous phase can be in the range of about 1:1 to about 1:10, or about 1:2 to about 1:6.

One useful aspect of micelle carriers is that they can be employed for the delivery of therapeutic agents without chemically modifying the agent. The structure of the polymers described herein can be tailored in order to enhance the properties of the micelles for therapeutic agent delivery. Such tailoring includes varying the amount and nature of amino acid side chain modifications, such as those described in the Examples below.

Micelles formed from the polymers disclosed herein allow for the PEG side-groups of the polymers to concentrated at outer portions of the micelles, referred to as the micelle corona. The micelle corona is therefore hydrophilic and allows for its incorporation into cells.

One advantage of micelle compositions includes their ease of storage and delivery. Micelle compositions can be lyophilized and reconstituted before intravenous administration. This allows for a lower risk of agent precipitation, which can in some cases lead to embolism formation. Micelle compositions are capable of long blood circulation, low mononuclear phagocyte uptake, and low levels of renal excretion. Also, micelle compositions have enhanced permeability and retention (EPR) to increase the likelihood of their encapsulated therapeutics reaching their targets, for example, tumors.

Tumors typically have high vascular density, as well as defective vasculature. Accordingly, high extravasation occurs and there may be impaired lymphatic clearance. The endocytosis and subsequent micelle disaggregation allows for the release of the encapsulated agent its delivery into the cell.

Polyplex micelles of various diameters can be prepared. In various embodiments, the unloaded or empty micelles can be prepared. In other embodiments, the resultant micelles can have average diameters of less than about 200 nm, or less than about 100 nm. In another embodiment, the micelles can have an average diameter of between about 55 nm and about 90 nm. In one embodiment, cumulant diameters of polyplex micelles can be about 70 nm to about 90 nm for polymer/pDNA complexes with N/P ratios of 1 to about 20.

The small size of polymeric micelles that have PEG coronas can help the micelle carrier to stay unrecognized, as self, in a biological system. Other advantages associated with nanoscopic dimensions of polymeric micelles include the ease of sterilization via filtration and safety of administration. The core of the micelles can take up, protect and retain biologically active agents, leading to improved solubility and stability of the agents in vivo, their controlled release, and overall reduced toxicity and attenuated pharmacokinetic interaction with other treatment agents.

Related polyplex micelles and their uses are described by Kanayama, Kataoka, and coworkers, ChemMedChem 2006, 1, 439-444, which is incorporated herein by reference. Other related technology is disclosed by Fukushima, Kataoka, and coworkers, J. Am. Chem. Soc. 2005, 127, 2810-2811, which is incorporated herein by reference. Additionally, photochemical transfection technology is disclosed by Kataoka and coworkers, J. Controlled Release 2006, 115, 208-215, which is also incorporated herein by reference. Other useful information on polyamides and micelle technology can be found in WO 2005/118672 (Lavasanifar and Kwon), and U.S. Patent Application Publication Nos. 2004/0005351 (Kwon et al.), 2004/0116360 (Kwon), 2006/0251710 (Kwon and Forrest), each of which is incorporated herein by reference.

Micelles can be suitably formulated into pharmaceutical compositions for administration to human subjects in a biologically compatible form suitable for administration in vivo. Accordingly, in certain embodiments, a pharmaceutical composition is provided that includes micelles as described herein, in admixture with a suitable diluent or carrier.

The compositions containing micelles can be prepared by known methods for the preparation of pharmaceutically acceptable compositions that can be administered to subjects, such that an effective quantity of the therapeutic agent within the micelles is combined in a mixture with a pharmaceutically acceptable vehicle. Suitable vehicles are described, for example, in Remington's Pharmaceutical Sciences (2003, 20^{th} Ed.), in The United States Pharmacopeia: The National Formulary (USP 24 NF19) published in 1999, and in the Handbook of Pharmaceutical Additives (compiled by Michael and Irene Ash, Gower Publishing Limited, Aldershot, England (1995)). On this basis, the compositions include, albeit not exclusively, solutions of the micelles in association with one or more pharmaceutically acceptable vehicles or diluents, and contained in buffered solutions with a suitable pH and iso-osmotic with the physiological fluids. In this regard, reference can be made to U.S. Pat. No. 5,843,456 (Paoletti and Maki). In one embodiment, the pharmaceutical compositions can be used to enhance biodistribution and drug delivery of therapeutic agents, such as drugs.

In accordance with the methods of the invention, the described micelles, may be administered to a subject in a variety of forms depending on the selected route of administration, as will be understood by those skilled in the art. The micelles of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A micelle may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the micelle of the invention may be incorporated with excipient and used in the form of ingestible tablets, buccal tablets, troches, capsules, elixirs, suspensions, syrups, wafers, and the like. A micelle may also be administered parenterally.

Solutions of a micelle can be prepared in water suitably mixed with suitable excipients. Under ordinary conditions of storage and use, these preparations can contain a preservative to prevent the growth of microorganisms.

The pharmaceutical forms suitable for injectable use include sterile aqueous solutions or dispersion and sterile powders for the extemporaneous preparation of sterile injectable solutions or dispersions. The form should be sterile and should be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compositions described herein can be administered to an animal alone or in combination with pharmaceutically acceptable carriers, as noted above, the proportion of which is determined by the solubility and chemical nature of the compound, chosen route of administration and standard pharmaceutical practice. In an embodiment, the pharmaceutical compositions are administered in a convenient manner such as by direct application to the infected site, e.g. by injection (subcutaneous, intravenous, etc.). In case of respiratory infections, it may be desirable to administer the micelles of the invention and compositions comprising same, through known techniques in the art, for example by inhalation. Depending on the route of administration (e.g. injection, oral, or inhalation, etc.), the pharmaceutical compositions or micelles or biologically active agents in the micelles of the invention may be coated in a material to protect the micelles or agents from the action of enzymes, acids, and other natural conditions that may inactivate certain properties of the composition or its encapsulated agent.

In addition to pharmaceutical compositions, compositions for non-pharmaceutical purposes are also included within the scope of the present invention, such as for diagnostic or research tools. In one embodiment, the biologically active agents or micelles comprising such agents can be labeled with labels known in the art, such as florescent or radio-labels or the like.

The present invention includes a delivery system that can be used to deliver biologically active agents or formulations or pharmaceutical compositions. In one embodiment, the invention includes the delivery of nucleic acids. In another embodiment, the invention includes delivery of biologically active agents by loading them into micelles comprising a amphiphilic core and a hydrophilic outer surface, thus improving their delivery in aqueous mediums, such as blood and body fluids. In other aspects, the invention includes the delivery of biologically active agents while reducing their toxicity profile. The invention also includes a method for reducing aggregation of the micelle delivery vesicles of the invention. As such, it provides for better biodistribution of biologically active agents resulting in decreased toxicity and/or improved therapeutic efficacy.

Another aspect of the invention includes a method of delivering biologically active agents to treat a disease, condition, or disorder in a subject in need thereof comprising administering an effect amount of an agent-loaded micelle to a subject. In one embodiment, the disease, condition or disorder is cancer or drug resistant cancers, infectious disease or an autoimmune disease.

The dosage of the micelles of the invention can vary depending on many factors such as the pharmacodynamic properties of the micelle, the biologically active agent, the rate of release of the agent from the micelles, the mode of administration, the age, health and weight of the recipient, the nature and extent of the symptoms, the frequency of the treatment and the type of concurrent treatment, if any, and the clearance rate of the agent and/or micelle in the subject to be treated.

One of skill in the art can determine the appropriate dosage based on the above factors. The micelles may be administered initially in a suitable dosage that may be adjusted as required, depending on the clinical response. For ex vivo treatment of cells over a short period, for example for 30 minutes to 1 hour or longer, higher doses of micelles may be used than for long term in vivo therapy.

The micelles can be used alone or in combination with other agents that treat the same and/or another condition, disease or disorder. In another embodiment, where either or both the micelle or biologically active agent is labeled, one can conduct in vivo or in vitro studies for determining optimal dose ranges, drug loading concentrations and size of micelles and targeted drug delivery for a variety of diseases.

### General Preparatory Methods

The polymers and compositions described herein can be prepared by any of the applicable techniques of organic synthesis and the related arts. Many such techniques are well known to the skilled artisan. Accordingly, many of the known techniques are elaborated in, for example, Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as March, J., Advanced Organic Chemistry, Third Edition, (John Wiley & Sons, New York, 1985); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999).

### Preparation of Polyamides

In one embodiment, a polyamide chain can be prepared by methods known to those of skill in the art. These and other methods, such as those provided in the Examples below, provide for the efficient synthesis of various polyamides. The amino acid side chains of the polyamides can then be modified, for example, by attaching hydrophilic groups, such as PEG chains, and PEG chain derivatives. Various linkers can be used to prepare polyamides with side groups that degrade under certain physiological conditions. For example, hydrazone linkers can be used to link PEG chains to the polyamide backbone. Hydrazone linkers provide the advantage of molecular stability at neutral pH, while allowing for the hydrolysis of PEG groups via hydrazone bonds in an acidic environment.

Also, in several embodiments the polyamide polymers of the invention are di-block copolymers. However, this concept can be varied by attaching additional blocks to one end or the other of this di-block, for example, to form a triblock polymer. The third block can be the same as one of the di-block groups, or it can be a different block, such as a physiologically acceptable poly(amino acid) of about 5 to about 150 repeating units. Additionally, the two blocks of the di-block polymers described herein can be separated by an organic linking group by methods well known to those of skill in the art. In other embodiments, one of the blocks of the polymer can be replaced with a octa-poly(arginine) block, or one or more octa-poly(arginine) units can be conjugated to the polymer through a hydrazide group to provide additional cell-penetrating peptide properties to the polymer.

It should also be noted that while some side chain modification reactions go to near completion (such as forming hydrazides), other side chain modification reactions afford much lower yields. The result is a somewhat random arrangement of side chains on a particular block that have been chemically modified, often in order to tune the properties of a micelle that can be formed from the polymer. Accordingly, it is unlikely that PEG groups and other similar groups are spaced equally throughout a polymer block.

The following Examples are intended to illustrate the above invention and should not be construed as to narrow its scope. One skilled in the art will readily recognize that the Examples suggest many other ways in which the present invention could be practiced. It should be understood that many variations and modifications may be made while remaining within the scope of the invention.

### EXAMPLES

### Example 1

### Synthesis and Evaluation of Biodegradable Poly(Amino Acid) Block-Copolymers

### 1. Introduction.

Poly(ethylene glycol) (“PEG”) reduces immunogenicity, protects against enzymatic degradation and improves tumor accumulation of conjugates when attached to various liposomes, enzymes, and polycations.¹⁻⁵ In vivo studies in mice have demonstrated that PEGylated-PEI25 complexes are taken into RES organs less than PEI25.⁶ However, PEG minimizes interactions with cells and can reduce transfection efficiency.⁷ Wagner et al. have recently shown already that pH-triggered deshielding of PEG from PLL resulted in better in vivo gene transfer in mice compared to polyplexes with covalent linkages of PEG.⁸ In their study, they linked PEG via a pH-sensitive hydrazone bond to poly(l-lysine) (PLL) and PEI-25.

It was hypothesize that through the use of a degradable poly(amino acid) block-co-polymer, one for condensing pDNA and the other for pH-sensitive linkage to PEG, it may be possible to study structure-property relationships, varying PEG size as well as block-copolymer sizes. One of the important aspect lies in the methodology through which the acid-labile PEG groups are attached to the polymer. The DNA condensing polymer block can be designed to interact with pDNA at physiologic pH. The second block containing hydrazine groups will remain exposed to the outer-particle environment at this pH; thus polymers containing PEG “brushes” for pre- and post-PEGylation of polyplexes can be prepared.

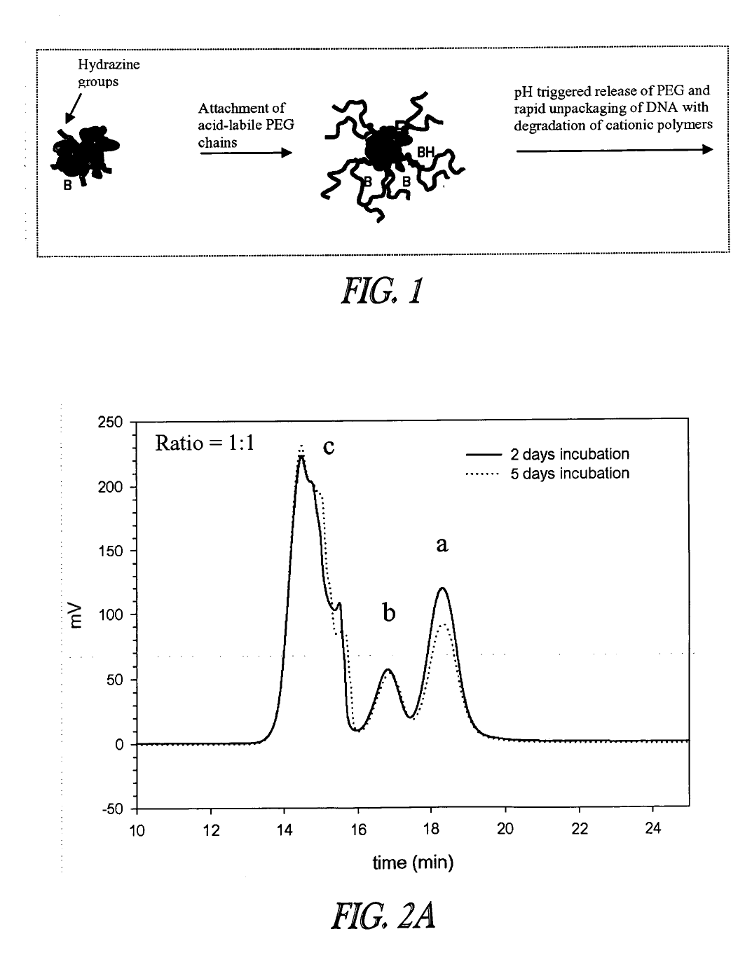

This approach will allow for the formation of more stable polyplexes for in vivo delivery because pre-PEGylation may hinder the formation of well-packed polyplexes for pH-sensitive release of PEG from polyplexes after endocytosis of the complexes. Furthermore, the pH-triggered release of PEG from complexes in endosomal compartments will aid in unpackaging the pDNA from the polymer and facilitate therapeutic agent, e.g., pDNA, escape into the cytoplasm. See for example, the schematic diagram shown in FIG. 1.

### 2. Experimental Procedures

### 2.1. Synthesis of the homopolymer poly(β-benzyl L-aspartate)

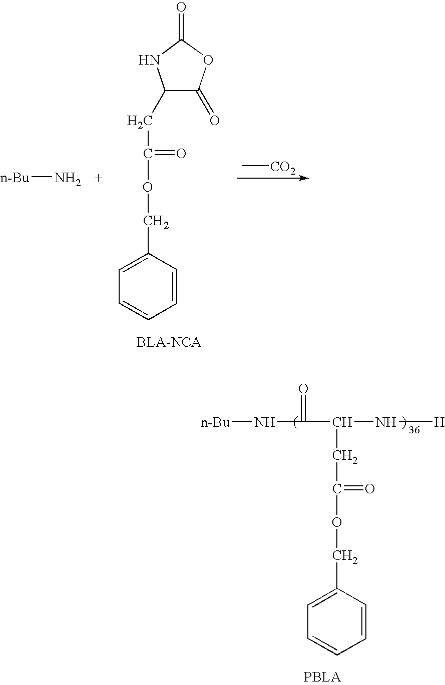

β-Benzyl L-aspartate N-carboxyanhydride (“BLA-NCA”) was synthesized by the Fuchs-Farthing method. Three grams of BLA-NCA (12 mM, MW=249 g/mol) was dissolved in an anhydrous cocktail mixture of 1:10 DMF to CH₂Cl₂ (40.5 mL CH₂Cl₂, 4.5 mL DMF) under vacuum. To the same flask, 340 μL of butylamine, 10× diluted with CH₂Cl₂ (MW=74.14 g/mol, d=0.74), was added to initiate polymerization of BLA-NCA. The flask was quickly moved to a 35° C. water bath with a stir bar to react for 24 hours.

Extent of polymerization was evaluated after 24 hours through IR spectral analysis, by monitoring the disappearance of three distinctive NCA peaks (1850 cm⁻¹: C═O asymmetric stretching; 1780 cm⁻¹: C═O symmetric stretching; 910 cm⁻¹: C═O stretching) and polymer purity was evaluated by GPC analysis (mobile phase: 10 mM LiCl in DMF).

The final product poly(β-benzyl L-aspartate), or “PBLA”, was precipitated from cold ether and the pellet of fine crystals was collected by centrifugation (10,000 RPM at 4° C., 10 minutes). After carefully pouring out the supernatant, a small amount of CH₂Cl₂ was added to the white crystal pellet to aid dissolution and the solution was transferred to a new flask where 100 mL of benzene was added for overnight freeze-drying. Degree of polymerization of PBLA was checked by ¹H-NMR measurement in DMSO-d₆ at 80° C. with respect to the methyl peak of butylamine (CH₃—: δ 0.8).

### 2.2. Preparation of the block-copolymers poly(β-benzyl L-aspartate)-poly(l-lysine(Z)) and poly(β-benzyl L-aspartate)-poly(l-lysine(TFA))

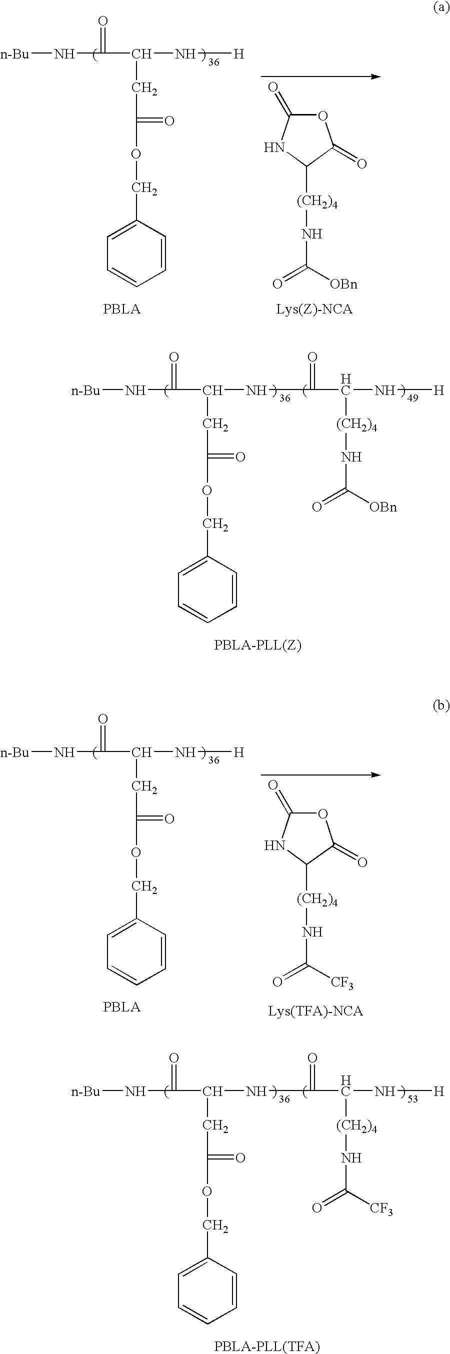

One gram of PBLA was used as an initiator for each block-copolymer to be synthesized (134 μM, DP=36, MW=7453 g/mol) and 15 mL of CH₂Cl₂ was added to the reaction flasks under vacuum. The PBLA polymer was allowed to completely dissolve at RT for 15 minutes to ensure complete chain extension into the solvent before the next step. In the case of Lys(Z)-NCA (scheme (a) above), 2.26 grams was added to 30 mL of dry DMF under vacuum (55 equiv, MW=306 g/mol) and the contents of the flask were immediately drawn with a syringe and added to the PBLA flask. The reaction was kept under an inert argon atmosphere.

Similarly, 1.98 grams of Lys(TFA)-NCA (scheme (b) above) was added to 30 mL of dry DMF (55 equiv, MW=268 g/mol) and immediately added to the second flask of PBLA. Both reactions were opaque in color and an additional 30 mL of CH₂Cl₂ was added to each flask to improve the solubility of both PBLA and Lys monomers. The flasks were moved to a 35° C. water bath with stirring to polymerize for 20 hours.

Extent of block-copolymer polymerization was checked via IR and purity of the block-copolymers was checked through TLC and GPC analysis. Both poly(P-benzyl L-aspartate)-poly(l-lysine(Z)) (PBLA-PLL(Z)) and poly(β-benzyl L-aspartate)-poly(l-lysine(TFA)) (PBLA-PLL(TFA)) reaction mixtures were evaporated under reduced pressure to remove excess CH₂Cl₂ before precipitating the polymers in a cold ether bath. The polymers were centrifuged to sediment the product and transferred to two new flasks for freeze-drying in benzene overnight. Degree of block-copolymer polymerization was measured by ¹H-NMR spectral analysis in DMSO-d₆ at 80° C. with respect to peak ratios of the butylamine methyl (CH₃—: δ 0.8) to three lysine methylene groups.

### 2.3. Preparation of poly(Asp-hydrazide)-poly(L-lysine(Z)) and poly(Asp-hydrazide)-poly(L-lysine(TFA))

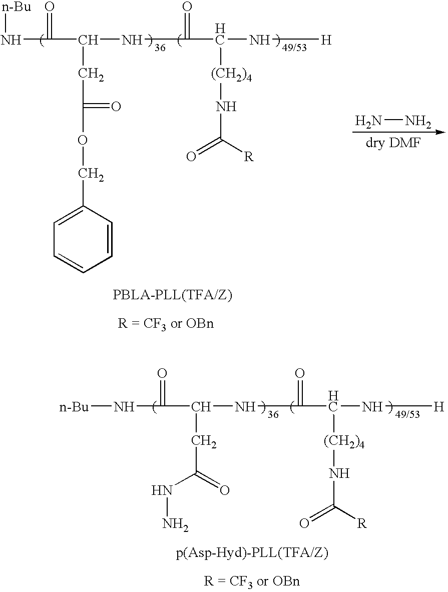

PBLA-PLL(Z) (500 mg) was dissolved in 10 mL of dry DMF in a reaction flask stored under vacuum. To the flask, 140 μL of anhydrous hydrazine (5 equiv, MW=32.05 g/mol) was added to the reaction. Similarly, 500 mg of PBLA-PLL(TFA) was dissolved in dry DMF in a separate flask and 155 μL of anhydrous hydrazine (5 equiv, MW=32.05 g/mol) was added. Both reactions were allowed to proceed for 3 hours in a 35° C. water bath. After 3 hours, p(Asp-Hyd)-PLL(TFA/Z) reactions were evaporated under reduced pressure to remove DMF and the polymers were ether precipitated in an ice bath. The polymers were centrifuged and the pellet was transferred to new flasks for overnight freeze-drying in benzene.

### 2.4. Acetylation of Heterobifunctional Primary Amines of Ace-PEG4K/7K-NH₂

¹H-NMR of Ace-PEG4k/7k-NH₂ in D₂O was obtained to confirm the molecular weight of each polymer. PEG4k molecular weight was determined to be 4K and PEG7k had a molecular weight of 7K. One gram of PEG4k/7k was weighted into a reaction flask under an inert argon atmosphere and dried under high vacuum for 30 minutes. Twenty mL of dry DMF was added to each reaction flask, for a final concentration of 50 mg/mL. Triethylamine (275 μL, 5 equiv, MW=101.19 g/mol, d=0.72) and 102 μL of acetic anhydride (3 equiv, MW=102 g/mol) were added to the PEG4k reaction flask. Similarly for PEG7k, 117 μL of triethylamine and 51 μL of acetic anhydride were added. The reactions were allowed to proceed for 1 hour in a 35° C. water bath, after which the polymers were ether precipitated, centrifuged to collect the polymers and freeze-dried in benzene overnight.

### 2.5. Activation of PEG4k/7K Through Deprotection of Acetal Groups

In separate reaction flasks, 500 mg of Ace-PEG4k/7k-COCH₃ was weighted and 4.5 mL of acetic acid and 0.5 mL of H₂O was added to each flask (10:1 AcOH to H₂O) for a final concentration of 100 mg/mL. The flasks were transferred to a 35° C. water bath for 5 hours with stirring. The polymers were ether precipitated to remove excess acetic acid and were filtered to collect the crystals. Acetonitrile was used to solubilize the polymer crystals one more time for another round of ether precipitation.

The final polymer crystals were filtered and collected and freeze-dried overnight in 50 mL of benzene. ¹H-NMR spectra in CDCl₃ at 25° C. of the polymers were taken to determine extent of acetal deprotection with respect to the peak ratios of the methylene protons of PEG (—OCH₂CH₂—: δ 3.5) and the aldehyde proton (CHO—: δ 9.8) introduced after acetal deprotection. Purity and quantitation of dimer formation after PEG activation was analyzed via GPC.

### 2.6. Pre-PEGylation of p(Asp-Hyd)-PLL(TFA) with CHO-PEG7K—COCH₃

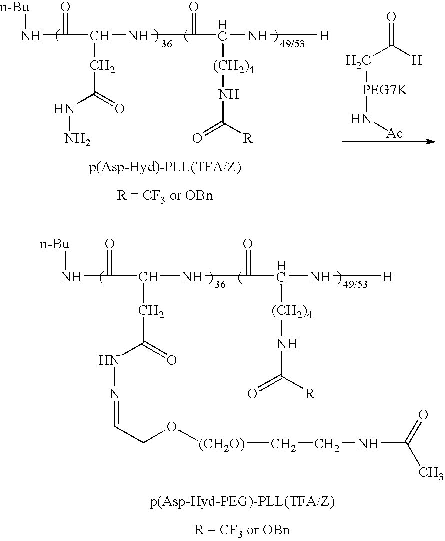

Varying molar ratios of 1:1, 1:0.5 and 1:0.1 p(Asp-Hyd)-PLL(TFA) to CHO-PEG7K-COCH₃ were reacted together in anhydrous DMSO. Assuming 100% hydrazine substitution of PBLA-PLL(TFA), 240 μL of p(Asp-Hyd)-PLL(TFA) (stock concentration 10 mg/mL in DMSO) was respectively reacted with 672 μL, 336 μL and 68 μL of activated PEG7k (stock concentration 50 mg/mL in DMSO). More DMSO was added to each of the 3 reaction vials for a total volume of 2 mL. The reaction vials were allowed to shake at RT for up to 5 days. GPC of each reaction was taken on day 2 and day 5 to monitor PEGylation progress of p(Asp-Hyd)-PLL(TFA).

### 3. Results and Discussion

### 3.1. Assessment of poly(β-benzyl L-aspartate)

Poly(β-benzyl L-aspartate), or “PBLA”, was prepared by the ring-opening polymerization of BLA-NCA initiated by the terminal —NH₂ of butylamine. The ratio of butylamine initiator to use was in proportion to the desired chain length of PBLA. In this case, theoretical calculations were made for a chain length of 35 and hence a 1:35 molar ratio of initiator to BLA-NCA was used. BLA-NCA is quite sensitive to moisture and it is important to seal the system under inert argon gas and/or store under vacuum to ensure good polymerization results. NCA's are hydrophobic molecules and the best solvent for solubilizing various types of NCA's will vary but in the case of BLA-NCA, a suitable solvent for the reactions was anhydrous CH₂Cl₂.

After 24 hours, an IR spectra of the final product was taken before proceeding further. At t=0 hours, there were three distinctive peaks present that are characteristic of the carbonyl groups on NCA; at 1850 cm⁻¹ a peak corresponding to asymmetric stretching of C═O, at 1780 cm⁻¹ a peak corresponding to C═O symmetric stretching and at 910 cm⁻¹ a peak corresponding to C═O stretching. After 24 hours, these three distinctive peaks disappeared, suggesting the end of polymerization, i.e., all BLA-NCA monomers were incorporated into the polymer.

To analyze the polydispersity of the newly synthesized PBLA, an RI detector on a GPC was employed. A single peak corresponding to the homopolymer PBLA appeared. GPC conditions employed (mobile phase: 10 mM LiCl in DMF at a flowrate of 0.8 mL/minute) were optimized for PEGylated polymers. It was expected that this hydrophobic homopolymer may interact differently with the column and would appear broader than PEGylated polymers. Nevertheless a calculated polydispersity index M_{w}/Mₙ of 1.197 was obtained with an estimated molecular weight of 7,829 g/mol according to calibration curves used.

The actual degree of polymerization obtained for PBLA was calculated by analyzing its ¹H-NMR spectra (DMSO-d₆ at 80° C.) and integrating peaks with respect to the methyl group present on PBLA that originated from butylamine (CH₃—: δ 0.8). From this integration, the peak intensity ratio of the phenyl protons on PBLA (δ 7.2) revealed the degree of polymerization of PBLA to be 36. This allowed calculation of an exact molecular weight of 7,453 g/mol for PBLA. From 3 grams of starting BLA-NCA, 2.204 grams of PBLA₃₆ were obtained, compared to a calculated theoretical yield of 2.4 grams (92% yield).

### 3.2. Assessment of poly(β-benzyl L-aspartate)-poly(l-lysine(Z)) and poly(β-benzyl L-aspartate)-poly(l-lysine(TFA))

Poly(β-benzyl L-aspartate)-poly(l-lysine(Z)) (“PBLA₃₆-PLL(Z)”) and poly(β-benzyl L-aspartate)-poly(l-lysine(TFA)) (“PBLA₃₆-PLL(TFA)”) were prepared in order to verify whether the deprotection approach of PLL has detrimental effects on reactivity of hydrazine towards PEG. PBLA₃₆-PLL(Z) requires deprotected under acidic conditions whereas PBLA₃₆-PLL(TFA) can be deprotected under more basic conditions. One goal was to synthesize acid-labile pre- and post-PEGylated complexes for gene transfection. Investigations as to whether it would be a better strategy to deprotect PLL after PEG attachment to hydrazine groups or before PEG attachment were desired. If PEG groups were attached via acid labile bonds to hydrazine groups and then deprotect PLL, the acidity could also cause PEG groups to be removed. However, if PLL is deprotected under acidic conditions first, the reactivity of hydrazine towards PEG in such acidic conditions may be affected.

The pKa of hydrazine is approximately 3-4 versus that of lysine, which is closer to 10. At lower pH (near endosomal pH of 4-5), both hydrazine and lysine terminal —NH₂ would be protonated and formation of a Schiff base with activated aldehyde-PEG would be hindered. As reaction conditions approach physiologic pH of 7.4 or higher, hydrazine moieties would being to deprotonate and only PLL should carry a charge on its terminal amine. This is ideal because it is desirable for PLL to condense DNA at physiologic pH, exposing the hydrazine groups. Hence, two types of PBLA₃₆-PLL(X) block-copolymers were prepared to investigate effects of deprotection on hydrazine reactivity to aldehyde-PEG.

PBLA was used to initiate the ring opening polymerization of NCA-Lys(Z) and NCA-Lys(TFA). A target degree of polymerization of 55 was attempted for both types of PLL chains and appropriate PBLA as initiator was used. The reaction was allowed to proceed for 20 hours at which point an IR spectra was taken to confirm that polymerization had ended. Once again, the three distinctive peaks characteristic of NCA C═O stretches (1850 cm⁻¹, 1780 cm⁻¹ and 910 cm⁻¹) were absent in the product spectra, suggesting that all NCA-Lys(TFA/Z) monomers have been incorporated into the product polymer.

GPC of both block-copolymers were obtained. Mobile phase used was 10 mM LiCl in DMF (0.8 mL/minute). Both PBLA₃₆-PLL(TFA) and PBLA₃₆-PLL(Z) chromatograms were not completely unimodal. However this was likely due to the mobile phase employed (DMF), which is not an optimal solvent for these new block-copolymers. Furthermore, these polymers are more hydrophobic than PEGylated polymers analyzed under similar conditions. The hydrophobic nature of these polymers may lead to more polymer interactions with the column, resulting in thinning chromatogram tails. Even though a few new peaks appeared in the chromatograms, they were all determined to be the same polymers in different conformations. To confirm that these additional peaks were not unreacted PBLA polymers, TLC analysis was conducted. Comparisons of the additional block-copolymer peaks with unreacted PBLA were conducted using a CH₂Cl₂ solvent system. No visible spots corresponding to PBLA were observed on the TLC plates for the block-copolymer aliquots.

Finally, to estimate degree of polymerization for PBLA₃₆-PLL(TFA), a ¹H-NMR spectra was taken in DMSO-d₆ at 80° C. by integrating with respect to the methyl group of butylamine (CH₃—: δ 0.8), followed by analysis of the peak intensity ratio of the three lysine methylene groups (—CH₂—: δ 1.26, 1.56, 1.95). For PBLA₃₆-PLL(TFA), the degree of polymerization was found to be 53. Similarly for PBLA₃₆-PLL(Z), a ¹H-NMR spectra was taken in DMSO-d₆ at 80° C. The degree of polymerization was determined by integration with respect to the methyl group of butylamine (CH₃—: δ 0.79), followed by analysis of the peak intensity ratio of the three lysine methylene groups (—CH₂—: δ 1.32, 1.92, 2.45). For PBLA₃₆-PLL(Z), the degree of polymerization was 49.

### 3.3. Assessment of Heterobifunctional Ace-PEG4K/PEG7K-NH₂ and Activation

Heterobifunctional (Diethyl)Acetal-PEG4k-NH₂ and (Diethyl)Acetal-PEG7k-NH₂ were analyzed for purity via GPC (mobile phase=10 mM LiCl in DMF) and unimodal peaks were observed for each. For Ace-PEG4K-NH₂, a PDI of 1.038 was obtained and for Ace-PEG7K-NH₂ a PDI of 1.027 was obtained. A ¹H-NMR spectra of each Ace-PEG4k/PEG7k-NH2 was taken in D₂O at 25° C. to determine actual molecular weight. PEG4k was found to be about 4K and PEG7k was found to be about 7K.

### 3.4. Assessment of Activated PEG4K/PEG7K

The ω-terminal amine of heterobifunctional poly(ethylene glycol) was acetylated and the acetal group was deprotected into an aldehyde to yield activated aldehyde PEG. Because the activated PEG is quite reactive, there is possibility of polymer coupling to form dimers. GPC of activated PEG4k and PEG7k, however, revealed almost unimodal curves. Mobile phase was 10 mM LiCl in DMF at a flowrate of 0.8 mL/min. From analysis of GPC runs of the activated PEG4k, about 4.8% of the reaction formed dimers and for activated PEG7k, about 2.1% formed dimers.

In order to verify acetal conversion into aldehyde, ¹H-NMR spectra of both activated PEG polymers were taken in CDCl₃ at 25° C. It was determined that for PEG4k, about 87% of acetal groups were converted to aldehyde functionalities. Degree of acetal conversion into aldehyde was calculated by integrating and measuring peak ratios of methylene protons of PEG to the aldehyde proton. For PEG7k, abut 91% of acetal groups were converted to aldehyde functionalities. Thus, activated PEG-aldehyde was successfully prepared in high yield.

### 3.5. Assessment of p(Asp-Hyd)-PLL(TFA) pre-PEGylation with Activated PEG7K

Poly(Asp-Hyd)-PLL(TFA) was pre-PEGylated at three different molar ratios of hydrazines to PEG7k. Assuming that all 36 monomers of PBLA reacted with hydrazine, equivalent molar ratios of PEG7k for 1:1, 1:0.5 and 1:0.1 were calculated. Several different organic solvents (CH₂Cl₂, DMF, AcN, DMSO, MeOH) were investigated before the pre-PEGylation reaction was carried out. None of the aforementioned organic solvents, except DMSO, was able to fully solubilize the block-copolymer. Despite any disadvantages of using DMSO (low volatility), it appeared to be a suitable solvent for carrying out the pre-PEGylation reaction. It is important that the block-copolymer be extended into a good solvent in order to increase level of PEG attachment. It is also favorable if the reaction between hydrazine and the PEG-aldehyde were to proceed quickly because long reaction periods may decrease the reactivity of the aldehyde-PEG.

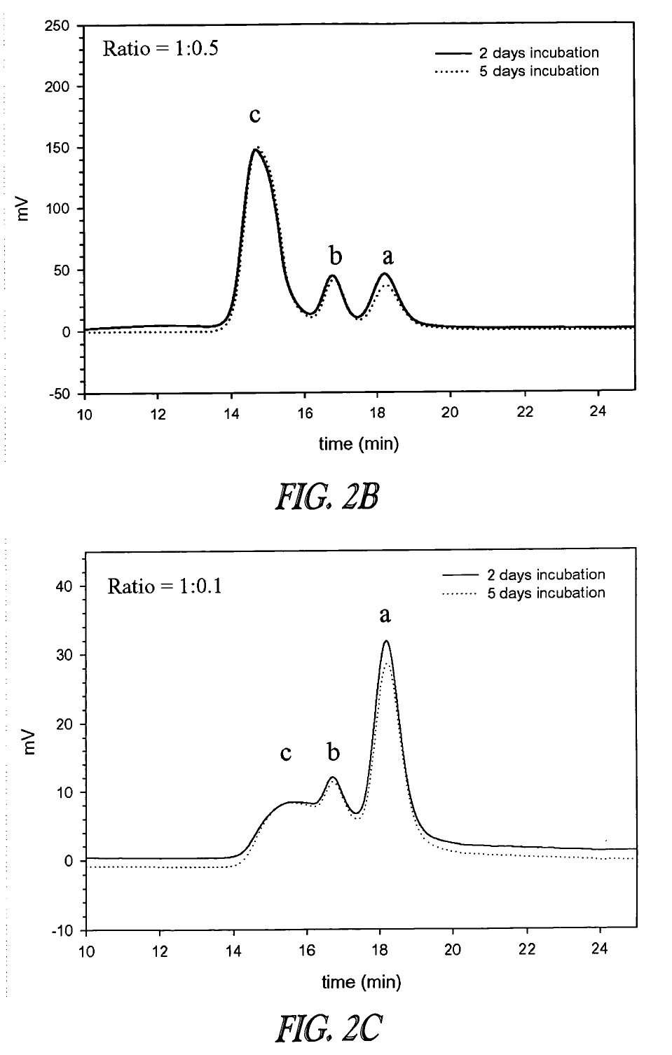

The reactions were allowed to proceed with shaking at RT for up to 5 days. A GPC of the reactions was taken at day 2 and day 5 to monitor extent of pre-PEGylation over time. The GPC solvent used was 10 mM LiCl in DMF, which was suitable but a more different solvent or solvent system may solubilizing these hydrophobic block-copolymers in a more optimal manner. During GPC analysis of p(Asp-Hyd)₃₆-PLL(TFA)₅₀ with aldehyde-PEG7k at a 1:0.1 molar ratio, block-copolymer appeared to interact with the column, either due to low PEGylation levels or due to lack of PEGylation from loss of PEG-aldehyde reactivity over this long reaction period, causing some decomposition (FIG. 2(c)). In contrast, molar ratios of 1:1 (FIG. 2(a)) reveals multimodal pre-PEGylated polymers (more PEG so less interaction with column) and molar ratios of 1:0.5 (FIG. 2(b)) reveals what appears to be a more unimodal distribution of PEG attachment to the block-copolymer. Despite waiting an additional three days after day 2, the reaction did not appear to proceed significantly further. This apparent lack of further reaction could be due to steric hindrance as more PEG groups become attached to the block-copolymer and/or because of loss of PEG-aldehyde reactivity.

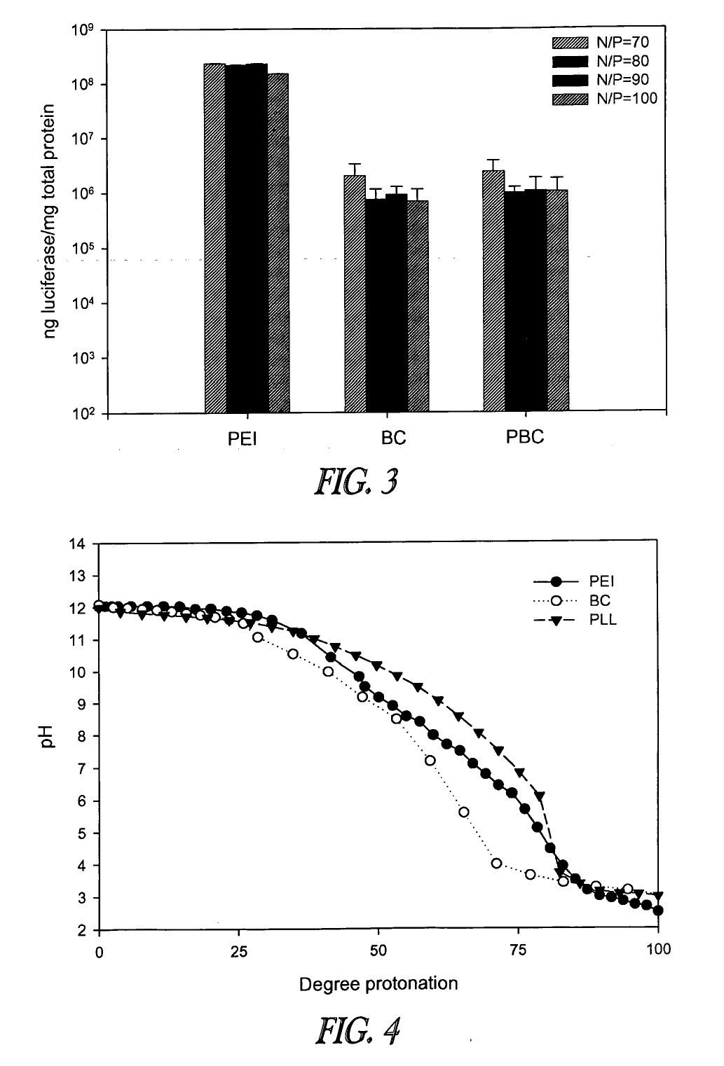

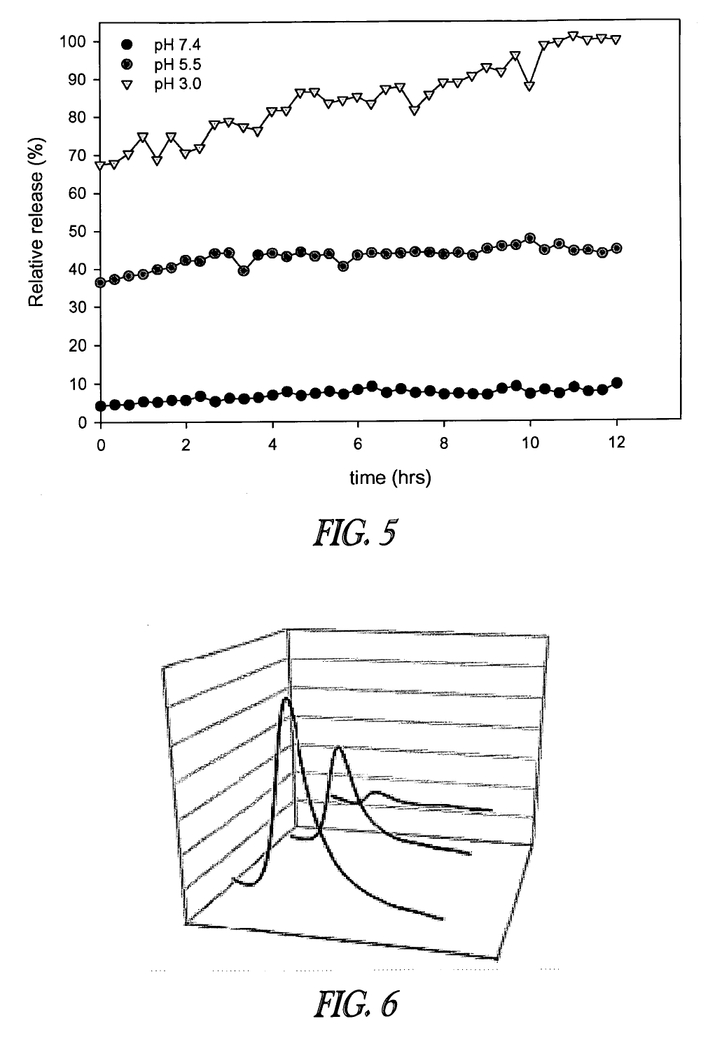

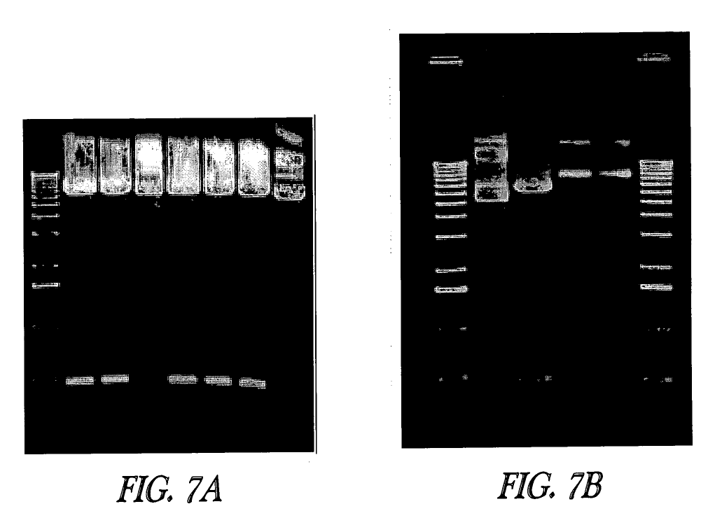

Thus, poly(Asp-Hyd)-poly(L-lysine(TFA)) and p(Asp-Hyd)-poly(L-lysine(Z)) have been successfully synthesized. It has been shown, using p(Asp-Hyd)-poly(L-lysine(TFA)), that hydrazide groups on a block-copolymer can interact with PEG-aldehydes to form an acid-sensitive Schiff bases (pre-PEGylation strategy for forming polyplexes). Formation of p(Asp-Hyd-PEG)-poly(L-lysine(TFA)) at three different molar ratios did not increase much beyond the second day of incubation due to steric hindrance from earlier PEG attachment and/or lost of aldehyde-PEG reactivity. The acid sensitivity of p(Asp-Hyd-PEG)-poly(L-lysine(TFA)) was analyzed by verifying release of PEG from the block-copolymer with GPC through the addition of various amounts of TFA and incubating overnight (FIG. 3).

### Example 1 Citations

1. Y. Kodera, et al. “PEGylation of proteins and bioactive substances for medical and technical applications.” Prog. Polym. Sci. 1998, 23:1233-1271.
2. K. D. Hinds and S. W. Kim, “Effects of PEG conjugation on insulin properties.” Advanced Drug Delivery Rev. 2002, 54:505-530.
3. M. Ogris et al. “Tumor-targeted gene therapy: strategies for the preparation of ligand-polyethylene glycol-polyethylene/DNA complexes.” J. Controlled Release 2003, 91(1):173-181.
4. S. Katayose and K. Kataoka K. “Water-soluble polyion complex associates of DNA and poly(ethylene glycol)-poly(L-lysine) block copolymer.” Bioconj. Chem. 1997, 8(5):702-707.
5. D. Papahadjopoulos et al. “Sterically stabilized liposomes: improvements in pharmacokinetics and antitumor therapeutic efficacy.” Proc. Natl. Acad. Sci. USA 1991, 88:11460-11464.
6. D. Fischer et al. “Effect of poly(ethylene imine) molecular weight and pegylation on organ distribution and pharmacokinetics of polyplexes with oligodeoxynucleotides in mice.” Drug Metab. and Disp. 2004, 32(9):983-992.
7. S Sung et al. “Effect of polyethylene glycol on gene delivery of polethylenimine.” Biol. Pharm. Bull. 2003, 26(4):492-500.
8. G. F. Walker et al. “Toward synthetic viruses: endosomal pH-triggered deshielding of targeted polyplexes greatly enhances gene transfer in vitro and in vivo.” Mol. Ther. 2005, 11(3):418-425.

### Example 2

### Poly(Asp-Hyd-PEG)-b-Poly(L-Lysine) for Gene Delivery of Yeast Cytosine Deaminase in GDEPT

The non-mammalian enzyme yeast cytosine deaminase (yCD) has shown potential in activating the prodrug 5-fluorocytosine (5FC) into cytotoxic 5-fluorouracil (5FU) in several tumor models. Unfortunately yCD's instability limits its bolus delivery in enzyme-prodrug type applications. A degradable poly(amino acid) block-copolymer poly(aspartatehydrazide)-poly(L-lysine) with acid-labile PEG attached through the formation of Schiff bases to hydrazide groups was synthesized. PEG provides ‘stealth properties’ to the complex at physiologic pH, minimizing immunogenic responses and prolonging systemic circulation. PEG release is pH-triggered from the polymer under acidic conditions (pH<7). The yCD gene was subcloned into the Hermes HRIgfp vector for regulation by a transactivator (RetroTet RTAb(+)) that binds to its promoter only in the presence of tetracycline. The results from this study provide valuable data supporting the low toxicity effects to cells in the presence of PEG and the importance of PEG release inside the cells for greater gene expression.

The non-mammalian enzyme yeast cytosine deaminase (yCD) has shown potential in activating the therapeutic prodrug 5-fluorocytosine (5FC) into cytotoxic 5-fluorouracil (5FU) in several tumor models. Unfortunately, yCD's instability limits its bolus delivery in prodrug-type applications. Therefore, this Example is directed to enhancing the delivery of the corresponding nucleic acid to the tumor site through a novel non-viral gene carrier system. The design of a pH-sensitive poly(aspartatehydrazide-PEG)-poly(L-lysine) block-copolymer addresses these issues.

A promising two-step therapy approach for tackling the shortcomings of chemotherapy was revived in 1987 by Bagshawe et al. and is generally known as enzyme-prodrug therapy (see Br. J. Cancer 1987, 56:531-532). The core idea behind enzyme-prodrug therapy is to deliver a specific enzyme that will synergistically activate an explicit prodrug in tumor cells. The nontoxic prodrug is systemically injected only after sufficient time has been allowed for the enzyme to localize at the tumor site. Only prodrug molecules in the tumor vicinity are activated, hence minimizing normal cell exposure to toxic drugs. The systemic injection of the gene corresponding to the enzyme in the form of a vector is called gene-directed enzyme prodrug therapy (GDEPT).

### In Vitro Characterization of yCD

The S. cerevisiae yeast cytosine deaminase (yCD) enzyme was cloned and expressed. The resulting 474 bp gene was subcloned into pET-15b to facilitate purification of the protein via a fused histidine tag and transformed into competent BL21(DE3) cells for protein expression. An estimate of about 25-30 mg of pure yCD-His was collected from a 1-liter culture. Thrombin cleavage of the histidine tag on yCD-His was achieved by incubating the protein for 2 hours at 23° C. in thrombin cleavage buffer. Enzyme activity characteristics for yCD and yCD-His was investigated using both the prodrug 5FC and the enzyme's natural substrate, the DNA base cytosine. Five-FC turnover rates remained within 10⁵ M⁻¹·s⁻¹ (Table 1) for both proteins (see E. Kievit et al. Cancer Res. 2000, 60:6649-6655). The diffusion controlled limit for extremely efficient enzymes and their substrates in solution is in the range 10⁸-10⁹ M⁻¹·s⁻¹, therefore it was concluded that yCD was a very efficient pretargeting enzyme for catalyzing 5FC to 5FU conversion, especially applicable to GDEPT. Table 1 shows enzyme activity of yCD and His-yCD, with average values ±SE, with the number of experiments shown in parentheses.

TABLE 1
Enzyme activity of yCD and His-yCD.

| | | | | k_{cat}/Kₘ |
|---|---|---|---|---|
| Enzyme | Substrate | Kₘ (mM) | k_{cat} (s⁻¹) | (M⁻¹ · s⁻¹) |
| yCD-HiS^{a} | 5-Fluorocytosine (6) | 1.0 ± 0.24 | 315 | 3.15 × 10⁵ |
| | Cytosine (4) | 4.04 ± 1.12 | 607 | 1.50 × 10⁵ |
| yCD^{b} | 5-Fluorocytosine (4) | 0.8 ± 0.18 | 448 | 5.60 × 10⁵ |
| | Cytosine (4) | 3.75 ± 0.69 | 741 | 1.98 × 10⁵ |

^{a}Amount of enzyme used was 10 uL drawn from a stock of 4.52 ug/mL
^{b}Amount of enzyme used was 10 uL drawn from a stock of 4.23 ug/mL

### Characterization of the Polymers

The PEG block-copolymer (PBC), e.g., poly(Asp-Hyd-PEG)-poly(L-lysine) of Example 1, successfully mediates gene delivery at about the same order of magnitude as the BC but shows dramatically less toxicity in C2C12 and MDA-MB-231 cell lines, especially evident at higher N/P ratios 70 to 100. FIG. 3 illustrates the transfection efficiency of PEI25, BC, and PBC at N/P ratios of 70-100 in MDA-MB-231 cells. The 5FU toxicity in MDA-MB-231 (drug concentration (μM) vs. % cell death) was analyzed by determining IC₅₀ values by fitting the data to a Hill-Slope curve: IC₅₀=2.9 uM (reported 0.9 μM on the National Cancer Institute website).

The BC buffers better at pH<7.0 than PLL alone due to the presence of hydrazide groups (FIG. 4). At physiologic pH, the hydrazone bond linking PEG to BC is stable and little PEG is released compared to pH 5.5 and pH 3.0 (FIG. 5). The release of PEG is instantaneous, resulting in BC aggregation which is used to monitor % release of PEG (FIG. 6). Thus, a block-copolymer with pH-sensitivity for PEG release has successfully been prepared. This will allow for the delivery and expression of high levels of yCD in target cells (e.g., cancer cells, such as colon cancer cells) followed by administration of 5FC (non-toxic) for conversion into toxic 5FU.

The yCD gene was subcloned into the Hermes HRIgfp vector for regulation by a transactivator (RetroTet RTAb(+) or RetroTet RtAb(−)) that will bind to its promoter only in the presence or absence of tetracycline (see A. M. Kringstein et al. Proc. Natl. Acad. Sci. USA 1998, 95, 13670-13675). This allows controlled studies of yCD expression in cells when looking at 5FC or 5FU toxicities.

FIG. 7 illustrates (A) the successful ligation of the yCD (˜500 bp) into the Hermes vector and screening of bacterial colonies, and that (B) propagation of the vector and digest was carried out to verify that the gene was inserted into the vector; lane 1=DNA ladder, lane 2=undigested Hermes-yCD, lane 3=digested HermesyCD, lane 4=lane 5=RtAb(−) vector.

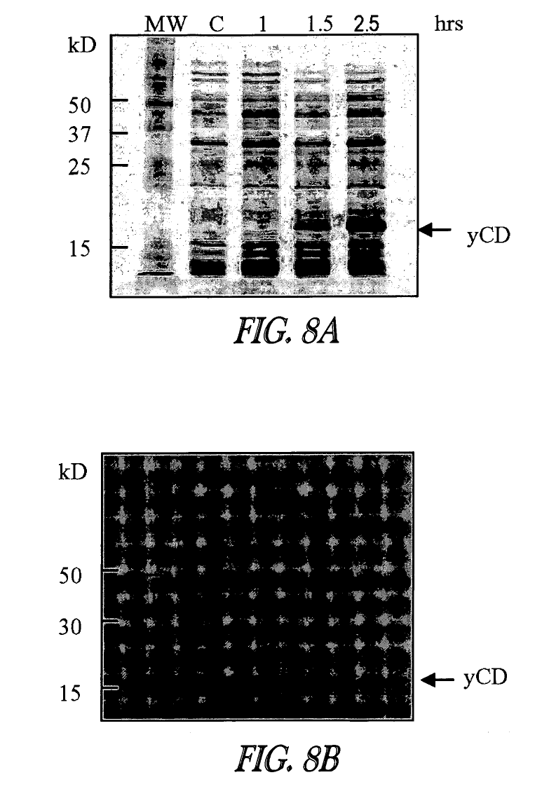

FIG. 8 illustrates (A) expression of yCD: Coomassie-stained gel of the crude lysate after induction of yCD at 28° C. with 0.4 mM IPTG showing, from left to right, crude protein extract, 1, 1.5, and 2.5 hrs after induction; and (B) a Western blot of crude protein extracts probed with Tetra-His conjugate at 1:1500 dilution followed by horse-radish peroxidase.

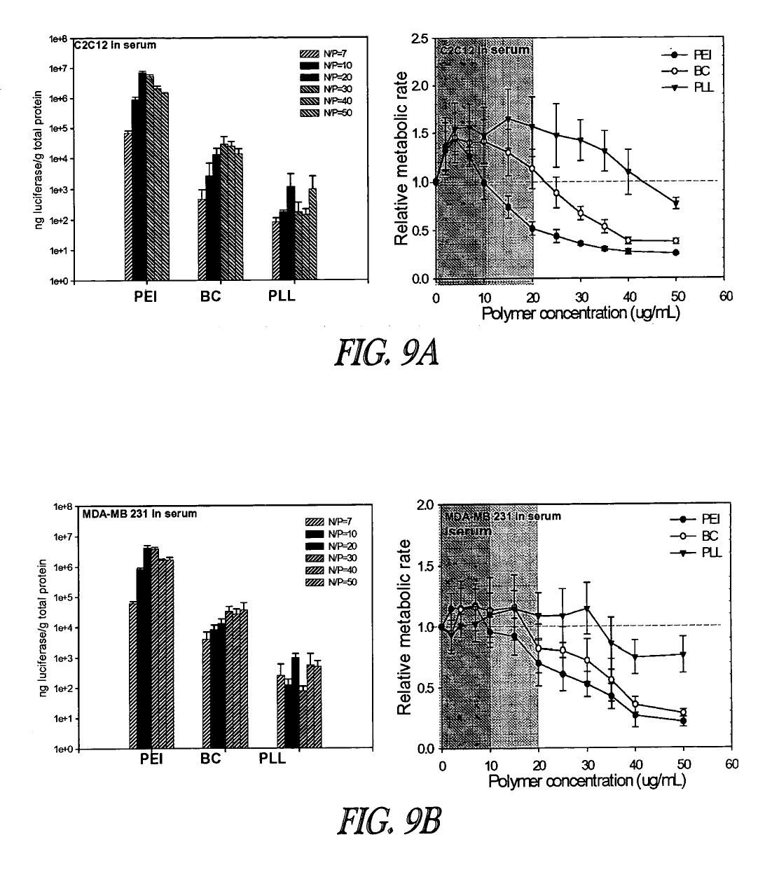

FIG. 9 illustrates transfection activity of PEI-25, the block copolymer (BC), and PLL at increasing equivalent N/P ratios and toxicity profiles of the free polymers in serum media for (A) C2C12; and (B) MDA-MB 231.

The results from this study provide valuable data supporting the low toxicity effects to cells in the presence of PEG and the importance of PEG release inside the cells for greater gene expression. The toxicity of 5FU and 5FC on various cell lines have also been confirmed.

### Example 3

### Poly(Asp-Hyd-PEG)-b-PLL Purification

An ion exchange column was used to purify the poly(Asp-Hyd-PEG)-b-PLL reaction to remove excess PEG. The ionic strength of the wash was kept low (50 mM phosphate, pH 8.0) with the flowthrough (unreacted PEG) being collected while the cationic polymer with PEG remains remained on the column. The polymer was collected by using a high ionic strength buffer (50 mM phosphate, 2 M NaCl, pH 8.0) and was dialyzed against 50 mM phosphate (pH 8.0) to remove the salt, before lyophilizing. NMR was used to estimate the average degree of PEG substitution on each polymer chain.

### Example 4

### Polyplex Formation with p(Asp-Hyd)-poly(L-lysine(TFA/Z))

Post-PEGylation can be carried out using variously protected block-copolymers or by deprotecting the block-copolymer first followed by formation of a polyplex. The block-copolymers may be suitably soluble in an aqueous environment once deprotected. Suitable solubility is advantageous because polyplexes form via electrostatic interactions with various therapeutic agents, e.g., DNA, which does not occur in an optimal manner in many organic solvent systems.

### Example 5

### pH-Responsive Multi-PEGylated Dual Cationic Nanoparticles Enable Charge Modulations for Safe Gene Delivery

### 1. Introduction.

The intelligent design and characterization of dynamic cationic nanoparticles formed from the self-association of a pH-responsive block copolymer is described in this Example. The platform allows pH-sensitive multilevel PEGylation and imparts nanoparticles with the ability to transition from neutral to charged at a pH of about 5.

In gene therapy, the cytotoxicity of many polycations is undesirable and has been attributed to nonspecific membrane destabilizing effects and intracellular polyplex-mediated toxicity. To help prolong the pharmacokinetic profile of nonviral vehicles for gene delivery, the cationic surface charge of current systems can be shielded through the conjugation of polyethylene glycol (PEG) chains to the particle surface. However, the design of an intelligent polycation with environment-sensing charge modulations is needed to minimize cytotoxicity and enhance gene expression. This Example describes the a novel di-cationic block copolymer, poly(aspartate-hydrazide)-block-poly(L-lysine), which is capable of pH-mediated endosomal membrane disruption based on charge interactions. The block copolymer has negligible toxicity elsewhere to the cell.

The poly(L-lysine) segment, with a high pKₐ value of ˜9.4, preferentially forms a poly-ion complex with the negative phosphate groups of pDNA, whereas the pH-responsive poly(aspartate-hydrazide) segment, with the comparatively lower pKₐ, ˜5.0, is characterized by a substantial fraction of unprotonated amino groups at physiological pH. As a consequence, complexation between such a polymer and pDNA leads to the formation of a two-layered nanoparticle. In particular, the nanoparticle possesses an unprotonated pH-responsive segment to serve as both a scaffold for acid-labile linkages of various moieties, such as aldehyde-PEG, and to transition from neutral to charged for disrupting endosomal membranes, and safely enhancing gene expression. This system supports an endosomal escape mechanism based on charge interactions rather than the proton-sponge effect, providing a new classes of nonviral vectors.

Intelligent biomaterials that can emulate natural viruses by adapting to their environment with minimal toxicity to the cell are highly desired for gene therapy. These synthetic vehicles are often internalized in many different cell lines through receptor-mediated endocytosis into clathrin-coated vesicles that fuse to form early endosomes (pH 7.4-6), which become late endosomes (pH 6.0-5.5), and eventually lysosomes (pH 5.0).^{[1-3]} Research to date has primarily emphasized gene delivery carriers equipped with endosomal buffering, also known as the proton sponge effect (PSE),^{[4]} to escape acidic endolysosomes for mediating gene expression.^{[5,6]} However, natural viruses are not equipped with endosomal buffering properties. Instead, they frequently exploit the decrease in pH to expose fusogenic domains that can disrupt the endosomal or lysosomal membrane, resulting in release of the virus into the cytoplasm. Similarly, endosomal membrane disruptions with charged surfaces may also play an important role in helping particles escape from endosomes.^{[7,8]}

Despite their positive attributes, many common polycations are cytotoxic due to nonspecific membrane destabilizing effects and intracellular polyplex-mediated toxicity.^{[9-12]} From these observations, it is postulated that the appreciable effect of charge interaction on endosomal escape would be observed even in the absence of the PSE by designing a pH-responsive polycation with dual functional pKₐ values (high and low) to emulate the escape mechanism used by natural viruses.

### 2. Results and Discussion

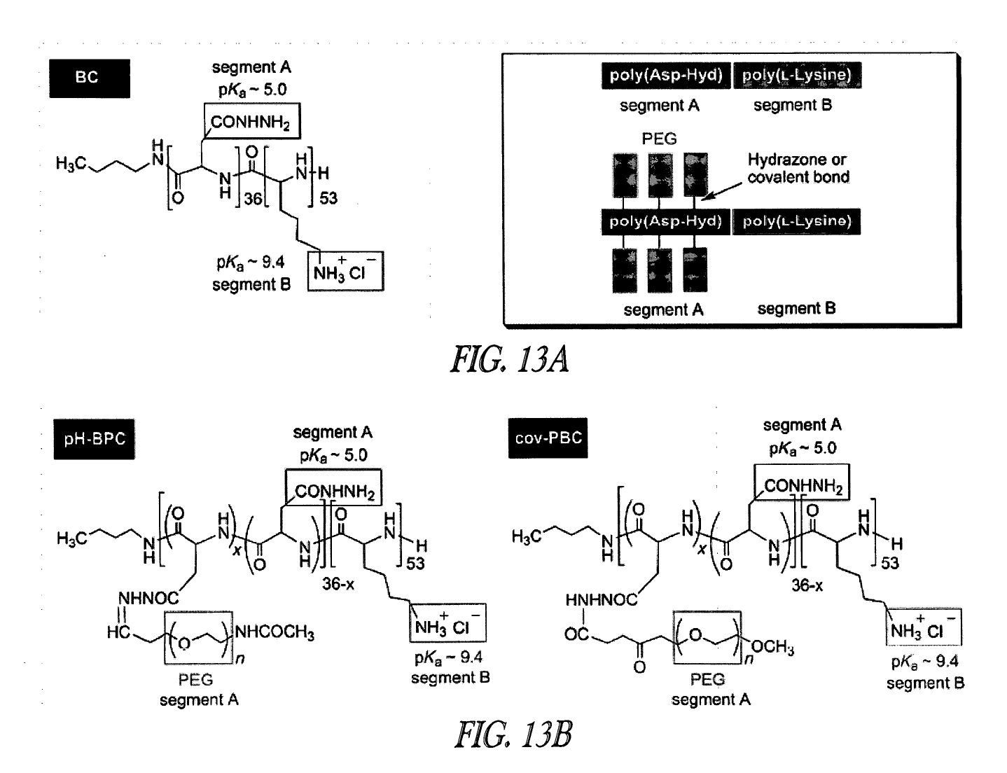

FIG. 13 shows the chemical structure of the dual-cationic block copolymer, poly(aspartate-hydrazide)-block-poly(L-lysine) (abbreviated BC), based on functional pKa differences between respective cationic blocks. The poly(L-Lys) (PLL) segment, with a high pKₐ value of ˜9.4, preferentially forms a poly-ion complex (PIC) with the negative phosphate groups of pDNA, whereas the pH-responsive poly(Asp-Hyd) segment, with a comparatively lower pKₐ value of ˜5.0, is characterized by a substantial fraction of unprotonated amino groups at physiological pH (FIG. 13a).

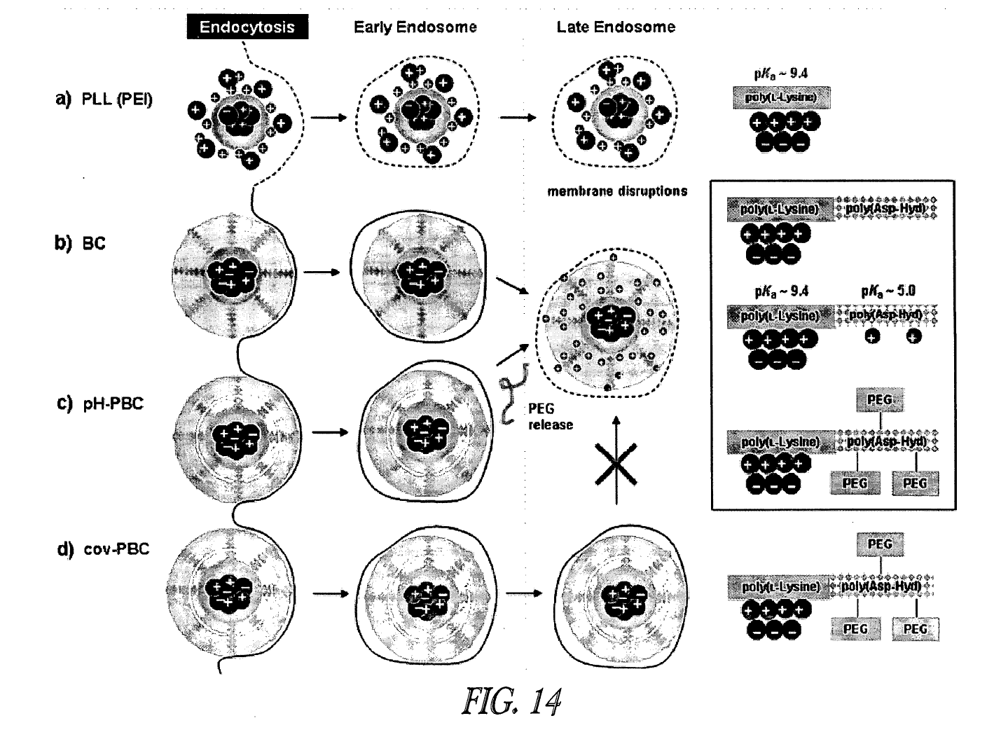

Some of these Asp-Hyd residues are coupled to aldehyde-PEG chains (ALD-PEG) through acid-labile hydrazone linkages to impart favorable stealth properties to the PIC (FIG. 13b). As a consequence, the complexation between such a polymer and pDNA may lead to the formation of a two-layered particle possessing an unprotonated pH-responsive poly(Asp-Hyd) segment that functions as both a scaffold for acid-labile linkages of various moieties, and that has the ability to undergo a transition from neutral to charged for disrupting endosomal membranes, as illustrated in FIG. 14. Therefore, the PIC described herein is formed from associations of such block copolymer chains: a) a pDNA condensing core made of PLL chains (known for not contributing to the PSE), and b) a shell composed of segments with repeating Asp-Hyd residues.

### 2.1 Chemical Synthesis

The backbone of the block copolymer is derived from poly(β-benzyl-L-aspartate)-block-poly(L-lysine) (PBLA-b-PLL; see Example 1). PBLA was prepared by the ring-opening polymerization of β-benzyl-L-aspartate N-carboxyanhydride (BLA-NCA), initiated by the terminal —NH₂ group of butylamine to yield a polymer with narrow distribution and degree of polymerization (DP) of 36. The terminal amine on PBLA was used to initiate ring-opening polymerization of lysine(TFA) N-carboxyanhydride monomers (Lys-NCA) to yield PBLA-b-PLL(TFA) with DP=53, in accordance with previous work in which a PLL length of ˜50 was determined optimal for gene expression.^{[5,13]}

Hydrazide groups were then conjugated with a substitution efficiency near 100 mol % on PBLA, using a procedure reported by Bae et al.,^{[14,15]} to give poly(Asp-Hyd)-b-PLL(TFA) (TFA=trifluoroacetyl). When deprotected, this polymer yields BC. Finally, ALD-PEG (M_{W}=7000 g mol⁻¹) was synthesized following procedures reported by Nagasaki et al.^{[16]} and characterized with ¹H NMR (CDCl₃; determined 91% acetal conversion into aldehyde groups) and gel permeation chromatography (GPC) (TSK-gel G3000PWXL and TSK-gel G4000PWXL; 10 mm LiCl in N,N-dimethylformamide (DMF); 0.8 mL min⁻¹; polydispersity index (PDI): 1.03).

The final PEGylated block copolymers [poly(Asp-Hyd-PEG)-b-PLL] had either about five ALD-PEG chains conjugated to each poly-(Asp-Hyd) block to form the acid-labile hydrazone linkage (pH-PBC), or about six COOH-PEG chains conjugated covalently by amide linkages to hydrazide groups to generate the non-hydrolysable control (cov-PBC), as shown in FIG. 13. There are substantial unconjugated hydrazide groups that remain on each block copolymer chain.

### 2.2. Physical Characterization

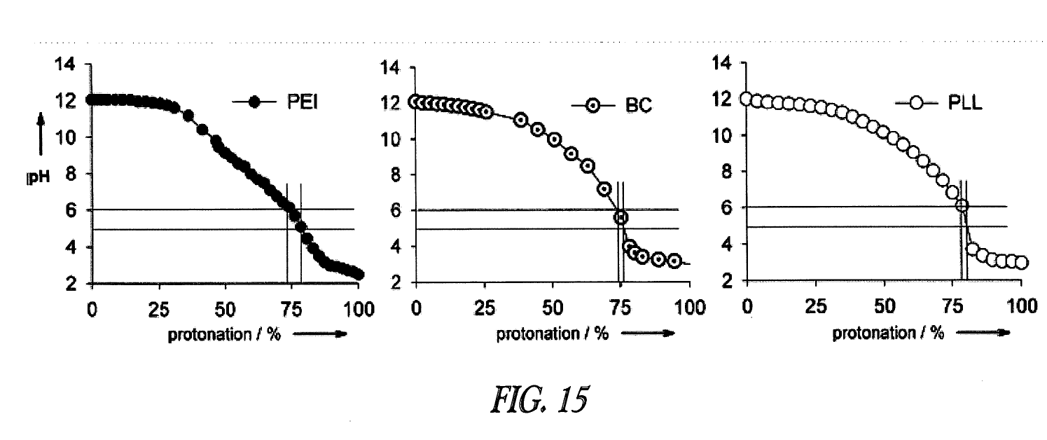

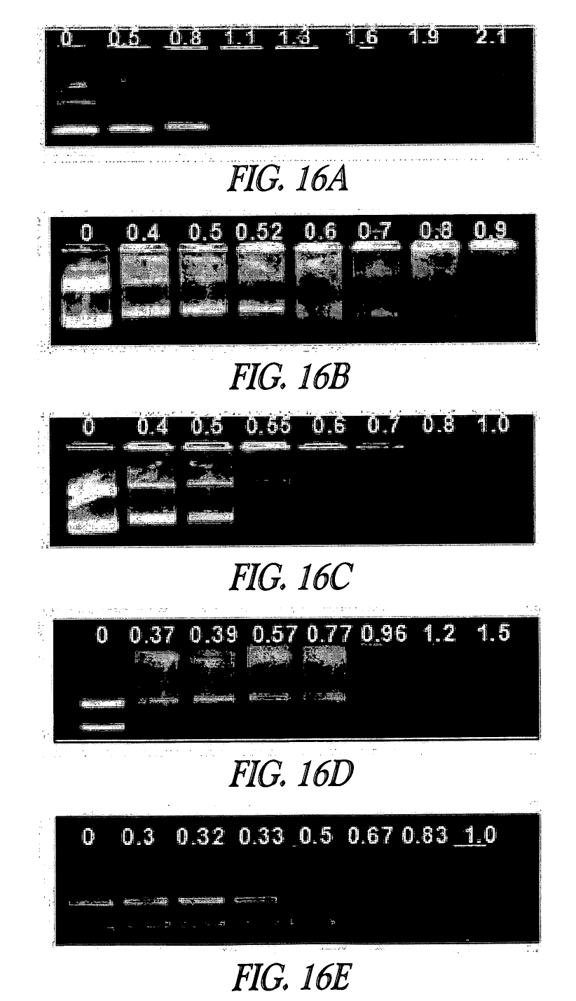

The protonation profile of BC reveals that hydrazide groups do not significantly buffer in the critical range known to be essential for PSE-mediated escape of endosomes (pH 5.0-6.0), similar to PLL (FIG. 15), and furthermore do not contribute significantly to pGL3 condensation at physiological pH, because mostly PLL groups undergo electrostatic interactions with pDNA (FIG. 16). This suggests that the PIC formed is composed of a PLL-DNA core with poly(Asp-Hyd) segments on the surface. Table 2 summarizes the mol % of various amines on three polymers; only side chain terminal amines are considered. The data indicate that the presence of hydrazide groups on BC does not contribute significantly to endosomal buffering in the critical range of pH 5.0-6.0 essential to the PSE.

TABLE 2
Mol % of amines in each polymer. Only terminal amines are
considered in the synthesized polymer.

| | MW | mol % | | |
|---|---|---|---|---|
| Polymer | (g/mol) | R₁—NH₂ | R₂—NH | R₃—N |
| commercial PEI | 25,000 | 32.3 | 38.0 | 29.7 |
| p(Asp-Hyd)₃₆-b-PLL₄₉ | 11,000 | 100 | n/a | n/a |
| commercial poly(L-lysine) | 9,200 | 100 | n/a | n/a |

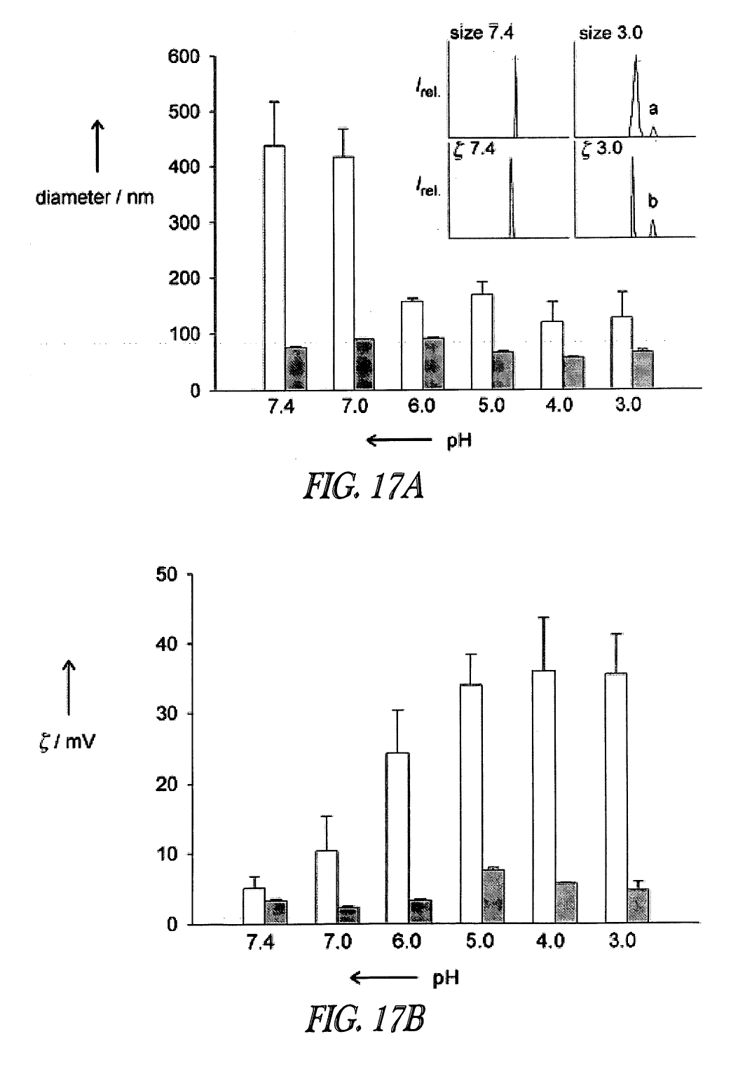

The effect of pH on colloidal properties of the PIC was also investigated (FIG. 17). Particles were formed at N/P=20 (to concur with the transfection experiments) with 2 μg pGL3 in water, incubated for at least 30 minutes, and diluted with low-ionic-strength buffers of appropriate pH before size measurement. The same sample was then injected into the instrument to obtain ξ potentials. At pH 7.4, BC particles are ˜440 nm with ξ potentials of ˜5 mV. At pH 3.0, BC particles are ˜100 nm with ξ potentials of ˜35 mV. For pH-PBC, the particles are ˜80 nm in diameter with a ξ value of ˜3 mV at pH 7.4. At pH 3.0, the average size is ˜70 nm and ξ ˜5 mV. However, pH-PBC profiles under acidic conditions (insert) point to particle instability as PEG is released. Large aggregates (peak a) with higher ξ values (peak b) appear (multimodal populations were observed for all acidic pH values). In contrast, cov-PBC had single population profiles (data not shown). (Note: the values reported were calculated by averaging the sums of peak intensities multiplied by magnitudes.)

At pH 7.4, PICs formed with BC have mostly unprotonated hydrazide groups exposed on the surface (˜5.0 mV), and counterion screening causes particles to exhibit aggregation behavior (diameter ˜440 nm). However, as hydrazide groups on PICs become protonated with decreasing pH, charged cationic surfaces are generated (˜35 mV), overcoming van der Waals interactions and resulting in electrostatic repulsions between individual particles (˜100 nm).

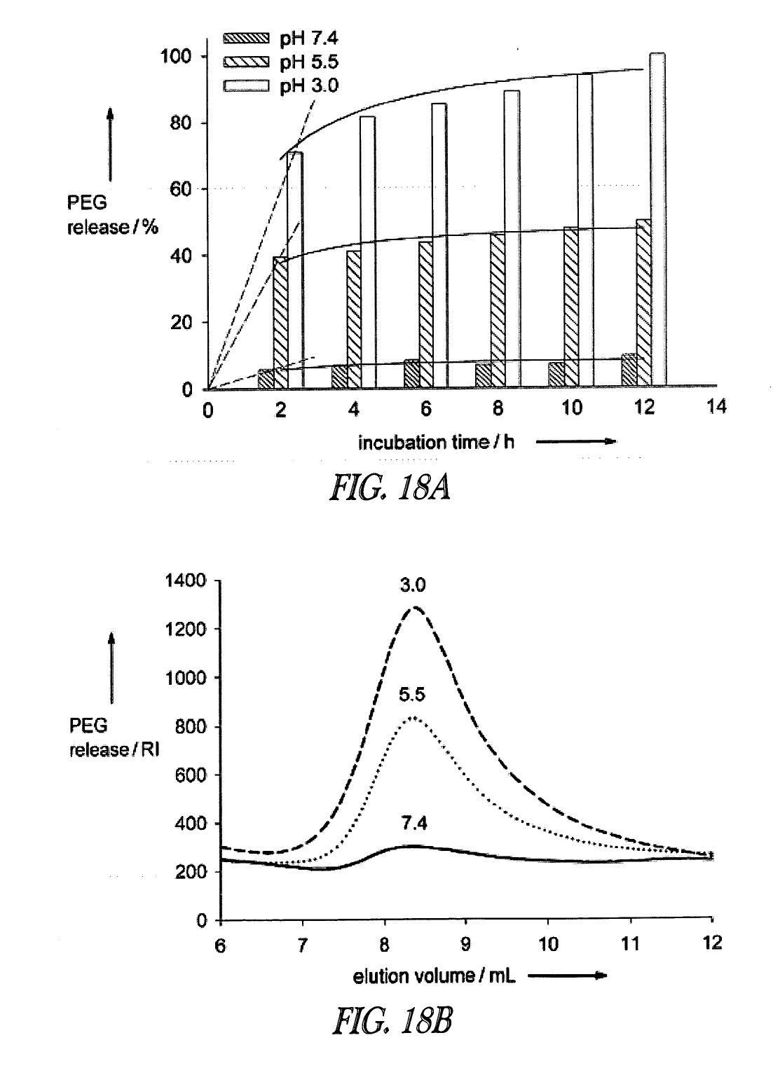

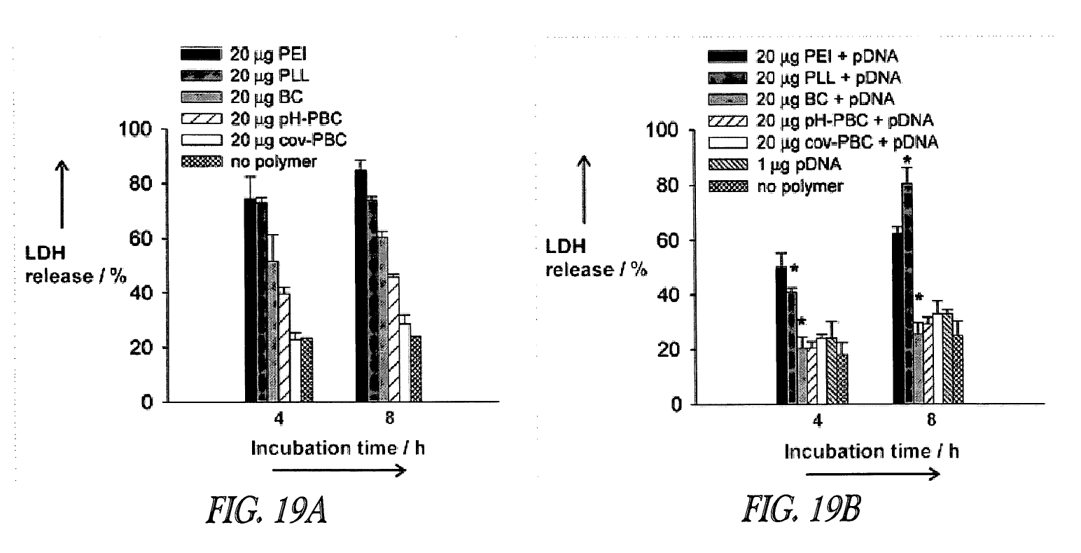

For pH-PBC, the size and ξ profiles are more informative than the averages reported (FIG. 17, insert). Although average ξ<8 mV and average diameter is <100 nm over the range of pH, the system is represented by distinct multimodal populations, indicative of reversible PEG release. This is in sharp contrast to the single population observed with cov-PBC under acidic conditions (data not shown). pH-Release studies confirm that, in a closed system, the hydrazone linkage is reversible and exhibits equilibrium behavior at various pH values over prolonged incubation (FIG. 18).

### 2.3. Biological Characterization

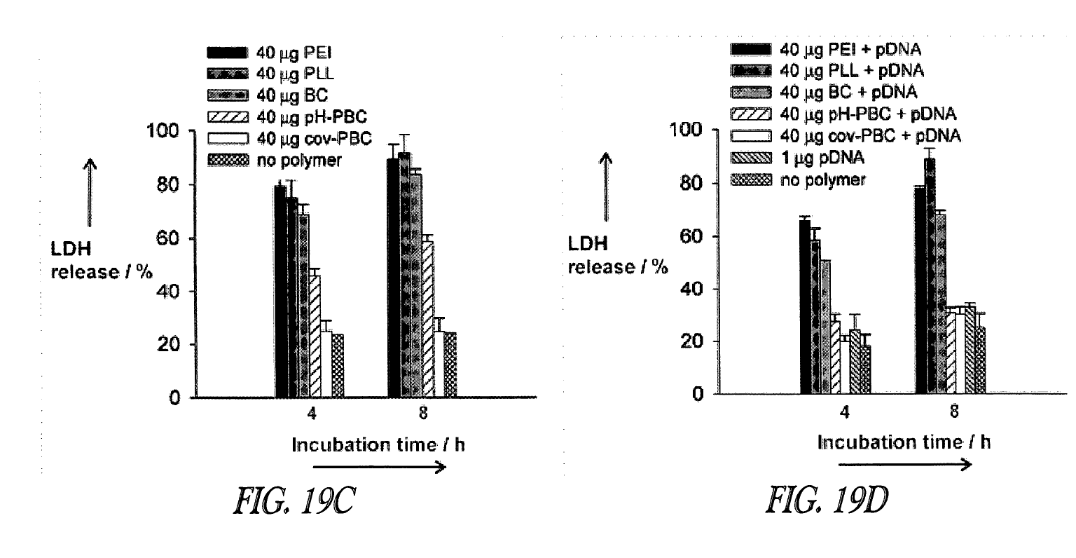

FIG. 19 illustrates membrane toxicity of polymers at concentrations of 20 and 40 μg mL⁻ toward MDA-MB-231 (human breast cancer) cells in DMEM serum-free media. Polymers were incubated both in free form [parts a) and c)] and also pre-complexed to 1 μg pGL3 [parts b) and d)]. Free polymers with cationic components (PEI, PLL, BC) are toxic to cells after just 4 hours of incubation. However, complexed polymers with concealed PLL (BC, pH-PBC, cov-PBC) effect significantly less LDH release at 20 μg than the static polycations (*p<0.05 using one-way ANOVA). At 40 μg, the presence of excess free polymers in solution with respect to pGL3 (see BC) re-establishes membrane toxicity.

Thus, the lactate dehydrogenase (LDH) release studies show that the dual-cationic polymer does not cause membrane toxicity when complexed with pDNA. At physiological pH, free PEI, PLL, and BC polymer chains have cationic properties that cause high levels of membrane toxicity to cells, relative to PEG-shielded polymers (FIG. 19a,c). However, if BC (20 μg mL⁻¹) is complexed with pGL3, the presence of many unprotonated hydrazides on the surface of the PIC causes negligible membrane toxicity (FIG. 19b). As BC concentration is increased in the presence of pGL3 (40 μg mL⁻¹), LDH release also increases, but that is most likely due to the presence of excess free polymers in solution with respect to pDNA concentration (FIG. 19 d). These results imply that freely accessible charged polymers cause membrane-associated toxicity, but that membrane damage is minimized when the cationic PLL block is concealed within the PIC core.

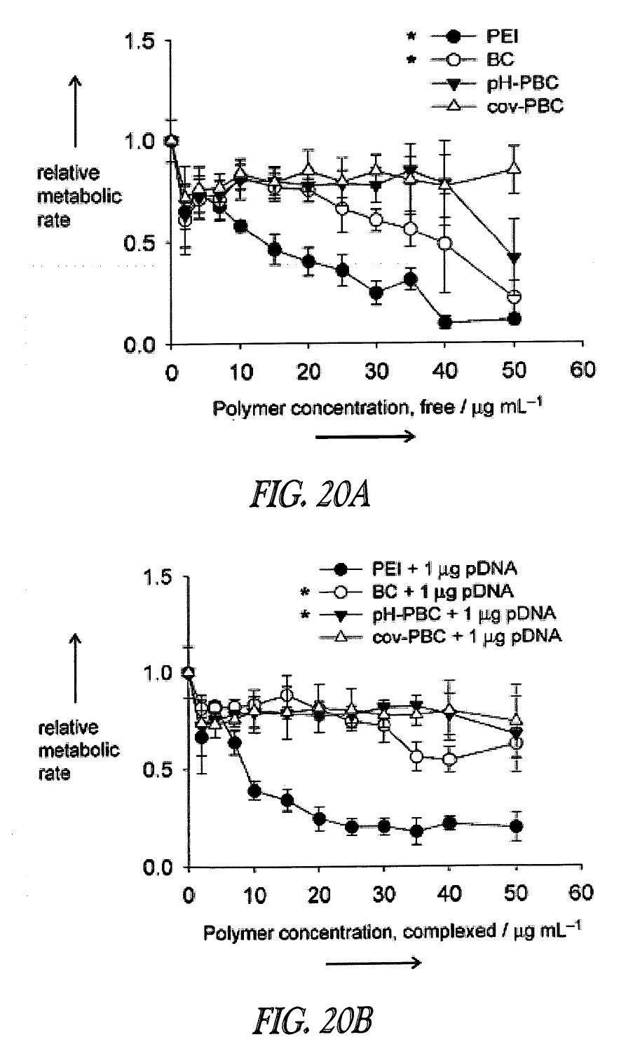

The metabolic activity of the polymers was then compared, both in free form and in complex with pGL3, to investigate the potential intracellular toxicity often associated with cationic polyplex-mediated uptake.^{[12]} The free polymers (PEI and BC) with accessible cationic components caused greater metabolic toxicity to the cell following internalization (FIG. 20a). PEG-shielded polymers exhibited low metabolic toxicity. On the other hand, PICs with concealed cationic PLL cores (BC, pH-PBC, and cov-PBC) gave little evidence of metabolic toxicity to the cell, in contrast to PEI-formulated particles (FIG. 20b). These results correspond well with the LDH assay data.

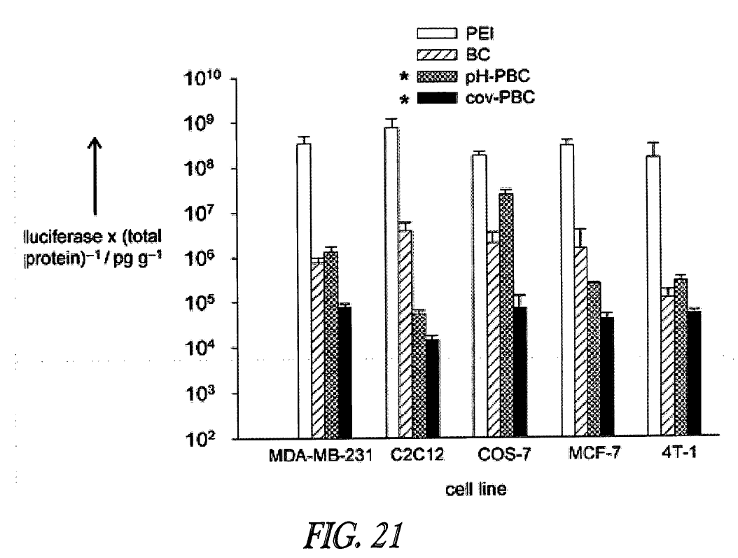

Finally, the transfection efficiency of these polymers was studied in five very different cell lines. It was found that pH-PBC and BC (both nontoxic) consistently resulted in higher gene expression relative to cov-PBC (FIG. 21) and PLL (data not shown). Note that the trend in transfection between the polymers and across different cell lines is not consistent. This might be a result of the fact that, in the absence of targeting ligands, various cell lines use different modes of particle internalization (such as clathrin and/or caveolae-mediated processes) that can ultimately affect uptake pathways and particle fate within the cell.^{[17-19]} Importantly, however, there is a consistent enhancement in gene expression for BC and pH-PBC compared with cov-PBC in the cell lines investigated. This demonstrates that a dynamic approach for release of PEG from BC is beneficial to increase endosomal membrane disruptions with charged particles, as the presence of PEG can minimize membrane interactions in the absence of charge.^{[20]}

It is of significant interest that the dual-cationic PICs described herein suggest an endosomal escape mechanism for polyplexes attributed to disruptions based on cationic-mediated cellular membrane interactions rather than through the PSE. In other words, selectively controlling interactions between polyplexes and the cell membrane is a new and facile approach for the design of effective and high-performance nonviral gene vectors. This results in nonviral vectors that are both safe to the cell while retaining high transfection efficiencies. Indeed, unlike PEI, which induces effective transfection activity but also causes high levels of nonspecific membrane-associated toxicity (FIG. 14a), neutral shielded particles (BC, pH-PBC, and cov-PBC) result in cellular uptake with minimal toxicity to the membrane (FIGS. 14b-14d).

Upon sensing a decrease in pH, PEG chains are released from pH-PBC PICs, and the remaining BC particle acquires favorable cationic properties for disrupting endosomal membranes. After the particles escape into the cytosol (pH 7.4), hydrazide groups start to deprotonate upon sensing the new pH, imparting neutral properties to the PIC again. This is very similar to how natural viruses respond to their environment by exposing a fusogenic domain at low pH and concealing it at higher pH, and which explains the low metabolic toxicity of PICs in the cell.

### 3. Conclusions

In summary, an polycation polymer with dual functional pKₐ values capable of safely emulating the escape mechanism used by natural viruses has been described herein. This novel dual-cationic block copolymer, poly(Asp-Hyd)-b-PLL, is pH-mediated to disrupt endosomal membranes based largely on charge interactions, and is therefore minimally toxic to the cell elsewhere. This is a mechanism separate from the traditional PSE followed by osmotic rupture of the vesicle. Furthermore, the significant presence of unconjugated hydrazides on the block copolymer can serve as a scaffold for easily incorporating many additional pH-sensitive functionalities, in addition to ALD-PEG, to the PIC to boost gene expression further. Examples of this include PEG-shielded fusogenic peptides for endosomal escape, nuclear localization signals (NLS), dynein-binding moieties, and additional masked targeting peptides for nuclear localization.^{[21,22]} Therefore, these findings offer a new platform for generating more effective and complex multifunctional biomaterials for increasing gene expression.

### 4. Experimental Section

General materials. Branched polyethylenimine (M_{W}=25000 g mol⁻¹) and poly(L-lysine) hydrobromide (M_{W}=9200 g mol⁻¹) were purchased from Sigma-Aldrich (Milwaukee, Wis., USA). All materials were used without further purification. The pDNA encoding firefly luciferase (pGL3, 5.3 kb) was obtained from Promega (Madison, Wis., USA), transformed into electrocompetent DH5α cells, propagated in LB broth (1 L) supplemented with ampicillin (100 μg mL⁻¹), and purified with a plasmid Maxiprep kit (BioRad, Hercules, Calif., USA). All pDNA had purity levels of 1.8 or greater by UV/Vis (A₂₆₀/A₂₈₀). For cell culture work, Dulbecco's modified Eagle's medium (DMEM), RPMI 1640, phosphate buffered saline (PBS), fetal bovine serum (FBS), trypsin-EDTA (0.25% trypsin, 2.21 mm EDTA in Hank's balanced salt solution (HBSS)) and penicillin/streptomycin were purchased from Cellgro (Mediatech, Herndon, Va., USA).

Physical characterization. pH titration measurements for polymers were obtained with an Orion micro-combination pH/sodium electrode (Thermo Electron Corporation, Waltham, Mass., USA; see Supporting Information for general procedures). Dynamic light scattering and ξ potential data were obtained with a nanoZS 90 or a Malvern 3000HS series zetasizer (Malvern Instruments, UK). Polymers were mixed to form PICs at N/P=20 by addition of polymer to an equal volume of pGL3 in Milli-Q water. The sample was allowed to incubate at room temperature for at least 15 minutes before dilution with the appropriate buffer ranging from pH 7.4 to pH 3.0. Particle diameters and ξ potentials were measured following dilution at each pH value.

Cellular LDH release studies. Polymer interactions with cell membranes were evaluated by monitoring LDH release from MDA-MB-231 human breast cancer cells by using the CytoTox 96 non-radioactive assay from Promega (Madison, Wis., USA). One-way ANOVA was used to statistically compare LDH data. MDA-MB-231 cells were seeded in 96-well plates at 25000 cellswell⁻¹. After 18 hours, the plates were washed with PBS (2×) and serum-free DMEM (90 μL) was added. Each 96-well plate was divided into two sections: maximum LDH and experimental LDH release.

Polymer (free or complexed to 1 μg of pGL3) was added to both sections appropriately and incubated with the cells for 4 or 8 hours. At the end of each incubation period, PBS was added to the experimental LDH wells (10 μL for volume correction) and 50 μL of the final supernatant was removed and put into a separate 96-well plate. Next, 10 μL of lysis solution (Triton-X, 9% v/v) was added to the remaining maximum release wells and incubated for 1 hour at 37° C. Then, 50 μL of the supernatants from these lysed cells were used as controls for determining maximum LDH release. LDH substrate (50 μL) was then added to the separate 96-well plates containing supernatants, covered with aluminum foil, and shaken at room temperature for 30 minutes before reading the absorbance (λ=492 nm).

Cellular metabolic toxicity studies. Metabolic toxicity was assessed through the resazurin dye (Sigma-Aldrich, Milwaukee, Wis., USA) by incubating the respective polymers (free or complexed with 1 μg pGL3) in serum-containing media and monitoring the toxic effects of the polymers on the metabolic rates of cells through reduction of the dye. Briefly, 96-well plates were seeded at 20000 cellswell⁻¹ and incubated for 24 hours. Free polymer at increasing concentrations of 0-50 μg mL⁻¹ was added to wells and incubated in serum-containing media for 4 hours before refreshing the media. After 18 hours incubation, 10 μL of resazurin dye (60 μm in PBS) was added to each well and incubated for 4 hours before measuring fluorescence (λₑₓ=560 nm, λₑₘ=590 nm). Cell viability is reported as relative metabolic rate with respect to controls without polymers. Two-way ANOVA was used to compare the data.

Cell transfection studies. MDA-MB-231 (human breast cancer), C2C12 (murine myoblast) and MCF-7 (human breast cancer) cell lines were obtained and cultured according to ATCC protocols. COS-7 cells (Green African monkey kidney) were obtained from David M. Lynn (University of Wisconsin). 4T-1 cells (murine colon cancer) were obtained from the Small Molecule Screening Facility Center (University of Wisconsin) and cultured in RPMI 1640 supplemented with 10% FBS and 1% penicillin/streptomycin.

The luciferase assay system was purchased from Promega. Luminescence was measured with a microplate Tropix luminometer (Applied Biosystems). Protein content was obtained using the DC protein assay kit from BioRad (Hercules, Calif., USA), absorbance was measured with the Spectramax 190 microplate reader (Molecular Devices, Sunnyvale, Calif., USA), and results were fit to a known protein calibration curve. Cells were seeded at either 300000 cells well⁻¹ in 6-well plates or 150000 cells well⁻¹ in 12-well plates and cultured for 24 hours. The next day, wells were aspirated, washed with PBS (2×1 mL), and appropriate medium for each cell line was added (supplemented with 10% FBS and 1% penicillin/streptomycin).

Complexes (2 μg pGL3well⁻¹) formed at the indicated N/P ratio (N/P=1 is defined as the minimum amount of polymer required to retard 1 μg of pGL3 on a 0.75% agarose electrophoretic gel) were added to wells 4 hours before refreshing the media. After 12 hours, plates were assayed with the Promega luciferase assay system. Luciferase relative light units (RLUs) were converted into units of concentration by using known luciferase concentration standards. One-way ANOVA was used to compare transfection data. A value of p<0.05 was considered statistically significant.

Experimental Details.

### Preparation of p(Asp-Hyd)-b-PLL(TFA)

β-benzyl L-aspartate N-carboxyanhydride (BLA-NCA) and N-carboxyanhydride lysine(TFA) (NCA-Lys(TFA)) monomers were synthesized by the Fuchs-Farthing method. Poly(β-benzyl L-aspartate) or PBLA was prepared by the ring-opening polymerization of BLA-NCA initiated by the terminal —NH₂ of butylamine. The ratio of butylamine initiator to monomer used was in proportion to the desired chain length of PBLA. In this case, theoretical calculations were made for a chain length of 35 and hence a 1:35 mole ratio of initiator to BLA-NCA was used. The final product poly(β-benzyl L-aspartate) or PBLA was precipitated in cold diethyl ether and the resulting pellet of fine crystals formed was collected by centrifugation and freeze-dried in benzene (100 mL) overnight. The degree of polymerization (DP) of PBLA was checked with ¹H-NMR in DMSO-d₆ at 80° C. by integrating the phenyl protons (—CH₂C₆H₅—: δ 7.3 ppm) with respect to the methyl peak of butylamine (CH₃—: δ 0.8 ppm) and found to be 36 with a polydispersity index (PDI) of 1.20 (TSK-gel G3000PWXL and TSK-gel G4000PWXL; 10 mM LiCl in DMF; 0.8 mL/min).

The terminal amine of PBLA was used to initiate the ring opening polymerization of NCA-Lys(TFA). A target DP of 55 was attempted for the PLL chain, and appropriate amounts of PBLA as initiator was used. The final composition of the block-copolymer was checked with IR, GPC and TLC. ¹H-NMR was taken in DMSO-d₆ at 80° C. by integrating with respect to the methyl group of butylamine (CH₃—: δ 0.8 ppm) and then looking at the peak intensity ratio of the three lysine methylene groups (—CH₂—: δ 1.26, 1.56, 1.95 ppm). For PBLA₃₆-PLL(TFA), the degree of polymerization was 53. Next, the benzyl groups of PBLA-PLL(TFA) were substituted with hydrazide groups. Hydrazine reactivity towards PBLA benzyl groups is quite efficient, yielding nearly 100 mol % substitution^{[14,15]}.

### Preparation of p(Asp-Hyd-Peg)-b-PLL

pH-sensitive coupling. p(Asp-Hyd)₃₆-b-PLL(TFA)₅₃ (MW 16,500 g/mol) was reacted in DMSO at the desired hydrazide to ALD-PEG (MW 7000 g/mol) ratio for 24 hrs. This was attempted both for 2 PEG chains/polymer and 5 PEG chains/polymer. After the reaction, poly(Asp-Hyd-PEG)-PLL(TFA)₅₃ deprotection was done under basic conditions to prevent acid-sensitive release of PEG chains from the block-copolymer. The deprotection method was done in MeOH/H₂O (20:1) using K₂CO₃ as a base catalyst. The polymer was slightly insoluble in MeOH/H₂O due to the hydrophobic PLL(TFA) and free hydrazide groups (despite presence of PEG) and DMSO was added as needed to improve the solubility of the block-copolymer. The reaction proceeded for 3 hrs under reflux at 60° C. The expected product would have a molecular weight of 51,500 g/mol, hence it was filtered against water 3× using Millipore Amicon Ultra centrifugal filter devices (MWCO 30,000 g/mol) and the filtrate containing the deprotected pH-sensitive polymer was lyophilized. Again, ¹H-NMR in D₂O as before revealed that either 2 or 5 PEG chains were conjugated to each polymer.

Covalent coupling. Standard DCC/DMAP/NHS chemistry was used to couple methoxy-capped carboxylic terminated PEG (COOH-PEG) (MW 7000 g/mol, Polymer Source, Montreal, Canada) to hydrazide groups on poly(Asp-Hyd)₃₆-b-PLL(TFA)₅₃. For a theoretical yield of 6 PEG chains/polymer (or 1:0.24 hydrazide to COOH-PEG), 297 mg COOH-PEG (42 μmol) and 109 mg of the polymer (MW 16,500 g/mol, 178 μmol hydrazides) were each dissolved in 4.5 mL of DMSO to stir until fully solubilized. Next, 432 mg DCC (50 equiv), 48 mg NHS (10 equiv) and 2.8 mg DMAP (0.2 equiv) were added to the polymer vial and stirred to dissolve fully before COOH-PEG solution was added (concentration 10 mg/mL). The reaction was stirred at room temperature overnight. The following day, the solution was diethyl ether precipitated and centrifuged to collect the pellet. The pellet was dissolved in 11 mL of 0.1N NaOH to deprotect TFA groups. DMSO was added to the reaction to increase solubility (since the solution became cloudy in appearance) and was stirred for 1 hr at RT. The reaction was again filtered 3× with water using spin-tubes as before (MWCO 30,000 g/mol, Millipore Centriplus filter tubes) and lyophilized. ¹H-NMR (Varian 400 MHz, D₂O) reveals that there are experimentally 6 PEG chains/polymer.

pH titration method. An initial pH measurement was taken with the micro pH electrode, then 5 μL increments of 0.1N HCl was titrated into the solution, and the pH response following each addition of HCl was measured (n=3). The pH response was plotted versus degree of protonation for each polymer, based on the change in H+ levels in solution and amount of polymer used:

$$\% protonation = \frac{Δ {H}^{+}}{moles {NH}_{x}} \cdot 100$$

$$where$$

$$Δ {H}^{+} = [ moles {H}^{+} added ] - [ moles {H}^{+} in solution ] \\ [ moles {H}^{+} in solution ] = ( {10}^{- pH} ) \cdot ( total volume in solution )$$

and assuming that initial pH=pKa of the amines on the polymer. The total percentage of protonable amines in this batch of commercial PEI was experimentally measured using an inverse gated decoupled ¹³C-NMR experiment to minimize NOE contributions, as described by von Harpe et al (Varian 500 MHz; D₂O; delay=30 sec; pw=90; T=30° C.)^{[23]}.

### Example 5 Citations

[1] M. L. Forrest, D. W. Pack, Mol. Ther. 2002, 6, 57-66.
[2] J. Rejman et al., Biochem. J. 2004, 377, 159-169.
[3] F. M. Brodsky et al., Annu. Rev. Cell Dev. Biol. 2001, 17, 517-568.
[4] O. Boussif et al., Proc. Natl. Acad. Sci. USA 1995, 92, 7297-7301.
[5] S. Fukushima et al., J. Am. Chem. Soc. 2005, 127, 2810-2811.
[6] M. Oishi et al., Bioconjugate Chem. 2006, 17, 677-688.
[7] Z. Y. Zhang, B. D. Smith, Bioconjugate Chem. 2000, 11, 805-814.
[8] G. F. Walker et al., Mol. Ther. 2005, 11, 418-425.
[9] J. P. Clamme, J. Azoulay, Y. Mely, Biophys. J. 2003, 84, 1960-1968.
[10] W. T. Godbey et al., Gene Ther. 1999, 6, 1380-1388.
[11] W. T. Godbey, K. K. Wu, A. G. Mikos, Biomaterials 2001, 22, 471-480.
[12] S. M. Moghimi et al., Mol. Ther. 2005, 11, 990-995.
[13] K. Itaka et al., Biomaterials 2003, 24, 4495-4506.
[14] Y. Bae, S. Fukushima, A. Harada, K. Kataoka, Angew. Chem. 2003, 115, 4788-4791; Angew. Chem. Int. Ed. 2003, 42, 4640-4643.
[15] Y. Bae et al., Mol. Biosyst. 2005, 1, 242-250.
[16] Y. Nagasaki et al., Bioconjugate Chem. 1995, 6, 231-233.
[17] J. Rejman, A. Bragonzi, M. Conese, Mol. Ther. 2005, 12, 468-474.
[18] K. von Gersdorff et al., Mol. Ther. 2006, 14, 745-753.
[19] C. Goncalves et al., Mol. Ther. 2004, 10, 373-385.
[20] M. P. Xiong, M. L. Forrest, A. L. Karls, G. S. Kwon, Bioconjugate Chem. 2007, 18, 746-753.
[21] E. Mastrobattista et al., Nat. Rev. Drug Discovery 2006, 5, 115-121.
[22] R. M. Sawant et al., Bioconjugate Chem. 2006, 17, 943-949.
[23] A. von Harpe et al., J. Controlled Release 2000, 69, 309-322.

### Example 6

### Enzyme/Prodrug Systems for GDEPT

This Example provides several combinations of enzyme/prodrug systems that can be used for GDEPT (Table 3). It is worthwhile to note that most clinical trials of GDEPT have utilized viral vectors as gene delivery vehicles, but the success of these enzyme/prodrug combinations could very well translate to other carriers, especially as progress is being made in the non-viral field of gene delivery.

HSV-tk/ganciclovir. The most common gene used for GDEPT is the Herpes Simplex Virus type-1 gene which codes for the thymidine kinase enzyme (HSV-tk)^{[21]}. When the gene is transfected into tumor cells, it renders them sensitive to the prodrug ganciclovir (GCV). This is the only GDEPT system that has reached Phase III clinical testing^{[22]} and is commonly investigated for treating malignant gliomas^{[23,24]}. Fusion proteins of GFP and HSV-tk reveal that the enzyme is localized mostly in the nuclei [25] Cells expressing HSV-tk (tk⁺) are capable of monophosphorylating GCV, which in turn gets triphosphorylated by cellular kinases, and induces cell death by incorporating into the DNA. It was found that the bystander effect of GCV was not due to passive diffusion but due to transfer through cellular gap junctions between cells^{[26]}. GCV appeared to work better during S phase of mitosis when the cells were actively dividing, therefore this property could be a drawback in slower dividing hypoxic regions of a tumor^{[22]}.

CD/5FC. Cytosine deaminase (CD) is involved in the pyrimidine salvage pathway of bacteria and yeast, and is not endogenous to humans. CD can convert the non-toxic antifungal agent 5-fluorocytosine (5FC) into toxic 5-fluorouracil (5FU). The drug inhibits synthesis of DNA or RNA when its metabolite is incorporated into each respective chain, and the drug can also irreversibly bind to thymidylate synthase (an enzyme critical for DNA synthesis). Fungal CD has superior turnover rates both for the DNA base cytosine and 5FC, compared to bacterial CD^{[16]}, but is reportedly less stable^{[27]}. Because 5FC and 5FU are small non-polar molecules, they can therefore freely diffuse across cell membranes, generating a good bystander effect. The toxicity of 5FU is not cell-cycle specific and the antitumor effect of CD/5FC has been demonstrated in a variety of tumor systems^{[22]}, Encouragingly, a colon cancer model revealed that when 4% of cells expressed CD, it was sufficient to give a 60% cure rate in tumor-bearing animals, compared to the HSV-tk/GCV system where 50% of the cells had to be expressing to give the same cure rate^{[28]}. There have been two phase I clinical trials of the CD/5FC system using viral vectors for treating colon cancer metastasis in the liver, and prostate cancer^{[22]}. The prostate clinical trial resulted in 2 out of the 16 patients being cancer-free 1-year after the treatment^{[29]}.

CE/CPT-11. Carboxylesterase (CE) can hydrolyze esters into acids and carbamates into alcohols. The enzyme is endogenously expressed as two major isoforms in the human liver, and is also present in tumor tissues^{[30]}. The rabbit liver analogue of CE is reported to be up to 1000-fold more efficient than human CE at converting the prodrug irinotecan (7-ethyl-10[4-(1-piperidino)-1-piperidino] carbonyloxy-camptothecin, CPT-11) into the potent topoisomerase I inhibitor SN-38 (7-ethyl-10-hydroxycamptothecin)^{[31-33]}. The SN-38 inhibitor binds to the topoisomerase I enzyme (which cleaves, unwinds and ligates DNA strands) and prevents the DNA from re-ligating after being unwound. CPT-11 demonstrates good bystander effects^{[34]}, which makes it suitable for GDEPT. The CE/CPT-11 conversion efficiency was evaluated in 59 patients undergoing surgery for treatment of liver or colorectal cancers in France to monitor prodrug conversion in normal vs. tumor tissues^{[24]}. In 12 out of 53 patients, a ratio>5 for prodrug conversion in the tumor with respect to normal tissue was observed. This system shows promise in GDEPT since successful delivery and expression of rabbit CE may further increase prodrug/drug conversion at the tumor site^{[33]}.

bGlu/HMR-1826. This is an enzyme found in prokaryotes and eukaryotes. It is widely distributed in human tissues, especially liver and spleen^{[35]}, and beta-glucuronidase (bGlu) is also expressed at higher levels in tumor lysosomes^{[15,36]}. The enzyme can hydrolyze beta-glucuronic linkages from glycosaminoglycans and is highly specific for the glucuronyl residue^{[37]}, hence the linker is useful in the design of various prodrugs such as doxorubicin (HMR-1826), paclitaxel or 5FU^{[33, 37-40]}. Most prodrugs tend to be cell permeable for conversion into the active drug inside the cell, where the enzyme is being generated. However, HMR-1826 is hydrophilic and therefore cell impermeable. To overcome this issue, it was reported that cells transduced by a secretable form of bGlu (to establish an extracellular prodrug-drug depot of activating enzymes) showed great promise at converting HMR-1826 into lipophilic doxorubicin (DOX) and the effector drug generated resulted in a strong bystander effect with enhanced antitumor activity^{[37,41]}.

NTR/CB1954. Most of the GDEPT studies with this enzyme have used the NfsB gene coding for nitroreductase (NTR) from E. Coli^{[42]}. The enzyme requires a flavin mononucleotide (a cofactor derived from vitamin B2) to efficiently reduce quinones and aromatic nitrogen groups into hydroxylamines^{[43]}. NTR has broad substrate specificity for nitrogen mustards and carbamates^{[44]}, making this enzyme attractive for the design and activation of a variety of prodrugs. CB1954 is a prodrug that is efficiently reduced by NTR into the corresponding hydroxylamine, which is further metabolized by cellular kinases into the toxic agent capable of crosslinking DNA. CB 1954 demonstrates good bystander effect^{[45]}. The first clinical trial for this system was reported in 2004 when 18 patients with liver or colorectal cancers were treated by direct intratumoral administration of a replicant-deficient adenovirus vector coding for NTR^{[46]}. The study revealed that high expression of NTR was well-tolerated by patients.

### Conclusions

GDEPT in cancer therapy has been clinically tested over the years using a variety of enzyme/prodrug combinations. Certain combinations appear to sufficiently kill cancer cells, and each has its advantages. However, they all aim to improve the therapeutic efficacy of a lethal drug by minimizing non-specific tissue toxicities. Despite some of these promising preclinical results with viral vectors, there still remain many obstacles that must be overcome before GDEPT becomes the treatment of choice for cancer therapy, namely the issue of the delivery vector, and ultimately levels of expression of the therapeutic gene. The micelles of the invention overcome these obstacles to provide a significantly improved method of delivering these drugs than current methods.

TABLE 3
Prodrug/enzyme combinations investigated in GDEPT

| Prodrug | LogP | Mechanism of action | Drug | LogP | Refs. |
|---|---|---|---|---|---|
| GCV | −2.07 | Inhibits DNA polymerization | — | −6.70 | [47-51] |
| 5-FC | −1.79 | Inhibits DNA/RNA synthesis | 5-FU | −2.17 | [16, 48] |
| CPT-11 | +1.54 | Causes ssDNA breaks (topo I) | SN-38 | +2.02 | [48, 52-54] |
| HMR-1826 | +1.53 | Prevents DNA ligation (topo II) | DOX | +0.36 | [38-40, 55, 56] |
| CB1954 | +1.28 | Cross-links DNA | — | −0.34 | [48, 57-60] |

| | |
|---|---|
| GCV | 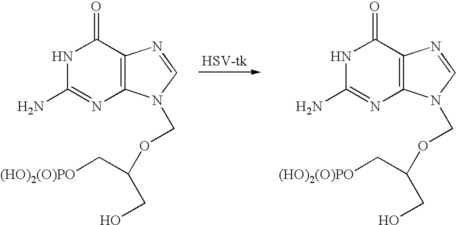 |
| 5-FC | 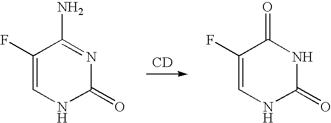 |
| CPT-11 | 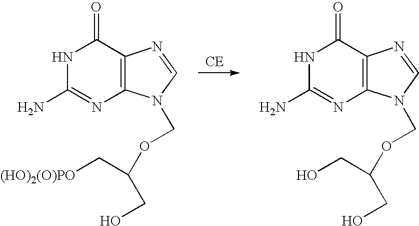 |
| HMR-1826 | 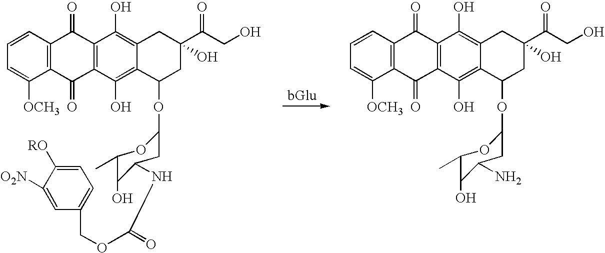 |
| CB1954 | 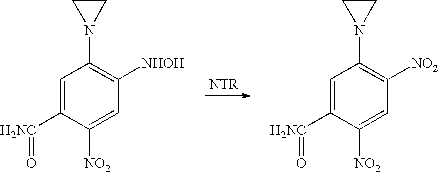 |

HSV-tk = thymydine kinase;
CD = cytosine deaminase;
CE = carboxylesterase;
bGlu = beta-glucuronidase;
NTR = nitroreductase

### Example 6 Citations

[15] Haisma et al. (1992) A monoclonal antibody-beta-glucuronidase conjugate as activator of the prodrug epirubicin-glucuronide for the specific treatment of cancer. British journal of cancer 66, 474-478.
[16] Kievit et al. (1999) Superiority of yeast over bacterial cytosine deaminase for enzyme/prodrug gene therapy in colon cancer xenografts. Cancer research 59(7), 1417-1421.
[21] Moolten, F. L. (1986) Tumor chemosensitivity conferred by inserted herpes thymidine kinase genes: paradigm for a prospective cancer control strategy. Cancer research 46(10), 5276-5281.
[22] Dachs et al. (2005) From bench to bedside for gene-directed enzyme prodrug therapy of cancer. Anticancer Drugs 16(4), 349-359.
[23] Smitt, P. S., Driesse, M., Wolbers, J., Kros, M., and Avezaat, C. (2003) Treatment of relapsed malignant glioma with an adenoviral vector containing the herpes simplex thymidine kinase gene followed by ganciclovir. Mol Ther 7(6), 851-858.
[24] Izquierdo et al, (1996) Human malignant brain tumor response to herpes simplex thymidine kinase (HSVtk)/ganciclovir gene therapy. Gene therapy 3(6), 491-495.
[25] Steffens et al, (2000) Enhanced green fluorescent protein fusion proteins of herpes simplex virus type 1 thymidine kinase and cytochrome P450 4B1: applications for prodrug-activating gene therapy. Cancer gene therapy 7(5), 806-812.
[26] Fick et al, (1995) The extent of heterocellular communication mediated by gap junctions is predictive of bystander tumor cytotoxicity in vitro. Proceedings of the National Academy of Sciences of the United States of America 92(24), 11071-11075.
[27] Katsuragi, T., Sonada, T., Matsumoto, K., Sakai, T., and Tonomura, K. (1988) Purification and some properties of cytosine deaminase from baker's yeast. Agric Biol Chem 53(5), 1313-1319.
[28] Trinh, Q. T., Austin, E. A., Murray, D. M., Knick, V. C., and Huber, B. E. (1995) Enzyme/prodrug gene therapy: comparison of cytosine deaminase/5-fluorocytosine versus thymidine kinase/ganciclovir enzyme/prodrug systems in a human colorectal carcinoma cell line. Cancer research 55(21), 4808-4812.
[29] Freytag et al, (2002) Phase I study of replication-competent adenovirus-mediated double suicide gene therapy for the treatment of locally recurrent prostate cancer. Cancer research 62(17), 4968-4976.
[30] Xu, G., Zhang, W., Ma, M. K., and McLeod, H. L. (2002) Human carboxylesterase 2 is commonly expressed in tumor tissue and is correlated with activation of irinotecan. Clin Cancer Res 8(8), 2605-2611.
[31] Rivory, L. P., Bowles, M. R., Robert, J., and Pond, S. M. (1996) Conversion of irinotecan (CPT-11) to its active metabolite, 7-ethyl-10-hydroxycamptothecin (SN-38), by human liver carboxylesterase. Biochemical pharmacology 52(7), 1103-1111.
[32] Guichard et al, (1998) Conversion of the CPT-11 metabolite APC to SN-38 by rabbit liver carboxylesterase. Clin Cancer Res 4(12), 3089-3094.
[33] Danks et al, (1998) Overexpression of a rabbit liver carboxylesterase sensitizes human tumor cells to CPT-11. Cancer research 58(1), 20-22.
[34] Kojima, A., Hackett, N. R., Ohwada, A., and Crystal, R. G. (1998) In vivo human carboxylesterase cDNA gene transfer to activate the prodrug CPT-11 for local treatment of solid tumors. The Journal of clinical investigation 101(8), 1789-1796.
[35] Holmberg, J. T., Hultberg, B., Hagerstrand, I., Ihse, I., and Isaksson, A. (1986) Tissue content and localization of lysosomal enzymes in cholestatic rats. Scandinavian journal of gastroenterology 21(2), 205-214.
[36] Bouvier, E., Schmidt, F., and Monneret, C. (2005) [Glucuronyl paclitaxel (Taxol) derivatives as tumor activated prodrugs]. Annales pharmaceutiques francaises 63(1), 53-62.
[37] Brusselbach, S. (2004) Extracellular beta-glucuronidase for gene-directed enzyme-prodrug therapy. Methods in molecular medicine 90, 303-330.
[38] Bakina, E., Wu, Z., Rosenblum, M., and Farquhar, D. (1997) Intensely cytotoxic anthracycline prodrugs: glucuronides. Journal of medicinal chemistry 40(25), 4013-4018.
[39] Guerquin-Kern, J. L., Volk, A., Chenu, E., Lougerstay-Madec, R., Monneret, C., Florent, J. C., Carrez, D., and Croisy, A. (2000) Direct in vivo observation of 5-fluorouracil release from a prodrug in human tumors heterotransplanted in nude mice: a magnetic resonance study. NMR in biomedicine 13(5), 306-310.
[40] de Bont et al, (1997) Synthesis and biological activity of beta-glucuronyl carbamate-based prodrugs of paclitaxel as potential candidates for ADEPT. Bioorganic & medicinal chemistry 5(2), 405-414.
[41] Heine, D., Muller, R., and Brusselbach, S. (2001) Cell surface display of a lysosomal enzyme for extracellular gene-directed enzyme prodrug therapy. Gene Ther 8(13), 1005-1010.
[42] Michael, N. P., Brehm, J. K., Anlezark, G. M., and Minton, N. P. (1994) Physical characterisation of the Escherichia coli B gene encoding nitroreductase and its over-expression in Escherichia coli K12. FEMS microbiology letters 124(2), 195-202.
[43] Zenno, S., Koike, H., Tanokura, M., and Saigo, K. (1996) Gene cloning, purification, and characterization of NfsB, a minor oxygen-insensitive nitroreductase from Escherichia coli, similar in biochemical properties to FRase I, the major flavin reductase in Vibrio fischeri. Journal of biochemistry 120(4), 736-744.
[44] Lovering, A. L., Hyde, E. I., Searle, P. F., and White, S. A. (2001) The structure of Escherichia coli nitroreductase complexed with nicotinic acid: three crystal forms at 1.7 A, 1.8 A and 2.4 A resolution. Journal of molecular biology 309(1), 203-213.
[45] Friedlos, F., Court, S., Ford, M., Denny, W. A., and Springer, C. (1998) Gene-directed enzyme prodrug therapy: quantitative bystander cytotoxicity and DNA damage induced by CB1954 in cells expressing bacterial nitroreductase. Gene Ther 5(1), 105-112.
[46] Palmer et al, (2004) Virus-directed enzyme prodrug therapy: intratumoral administration of a replication-deficient adenovirus encoding nitroreductase to patients with resectable liver cancer. J Clin Oncol 22(9), 1546-1552.
[47] De Clercq et al, (2001) Acyclic/carbocyclic guanosine analogues as anti-herpesvirus agents. Nucleosides, nucleotides & nucleic acids 20(4-7), 271-285.
[48] Denny, W. A. (2003) Prodrugs for Gene-Directed Enzyme-Prodrug Therapy (Suicide Gene Therapy). J Biomed Biotechnol 2003(1), 48-70.
[49] Tong et al. (1998) Improvement of gene therapy for ovarian cancer by using acyclovir instead of ganciclovir in adenovirus mediated thymidine kinase gene therapy. Anticancer research 18(2A), 713-718.
[50] Hasenburg et al. (1999) Thymidine kinase (TK) gene therapy of solid tumors: valacyclovir facilitates outpatient treatment. Anticancer research 19(3B), 2163-2165.
[51] Cheng, Y. C., Chadha, K. C., and Hughes, R. G., Jr. (1977) Biochemical and immunological characterization of deoxythymidine kinase of thymidine kinaseless HeLa cells biochemically transformed by herpes simplex virus type. Infection and immunity 16(2), 486-492.
[52] Wierdl et al. (2001) Sensitization of human tumor cells to CPT-11 via adenoviral-mediated delivery of a rabbit liver carboxylesterase. Cancer research 61(13), 5078-5082.
[53] Farquhar, D., Cherif, A., Bakina, E., and Nelson, J. A. (1998) Intensely potent doxorubicin analogues: structure-activity relationship. Journal of medicinal chemistry 41(6), 965-972.
[54] Tanaka, M., Iio, T., and Tabata, T. (1987) Purification and characterization of a carboxylesterase from rabbit liver lysosomes. Journal of biochemistry 101(3), 619-624.
[55] Rooseboom, M., Commandeur, J. N., and Vermeulen, N. P. (2004) Enzyme-catalyzed activation of anticancer prodrugs. Pharmacological reviews 56(1), 53-102.
[56] Jefferson, R. A., Burgess, S. M., and Hirsh, D. (1986) beta-Glucuronidase from Escherichia coli as a gene-fusion marker. Proceedings of the National Academy of Sciences of the United States of America 83(22), 8447-8451.
[57] Anlezark, G. M., Melton, R. G., Sherwood, R. F., Coles, B., Friedlos, F., and Knox, R. J. (1992) The bioactivation of 5-(aziridin-1-yl)-2,4-dinitrobenzamide (CB1954)—I. Purification and properties of a nitroreductase enzyme from Escherichia coli—a potential enzyme for antibody-directed enzyme prodrug therapy (ADEPT). Biochemical pharmacology 44(12), 2289-2295.
[58] Anlezark et al., (1995) Bioactivation of dinitrobenzamide mustards by an E. coli B nitroreductase. Biochemical pharmacology 50(5), 609-618.
[59] Mauger et al., (1994) Self-immolative prodrugs: candidates for antibody-directed enzyme prodrug therapy in conjunction with a nitroreductase enzyme. Journal of medicinal chemistry 37(21), 3452-3458.
[60] Zenno et al. (1996) Biochemical characterization of NfsA, the Escherichia coli major nitroreductase exhibiting a high amino acid sequence homology to Frp, a Vibrio harveyi flavin oxidoreductase. Journal of bacteriology 178(15), 4508-4514.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A polyamide polymer comprising amino acid units derived from amino acids with side chains that can have positive, neutral, or negative charges;
wherein at least one side chain of an amino acid of the polyamide is covalently linked to a poly(ethylene glycol) chain through a hydrazide moiety, and
wherein the hydrazide moiety is linked to the poly(ethylene glycol) chain at the N′ nitrogen of the hydrazide through a hydrazone bond.

2. The polymer of claim 1 wherein the amino acid units comprise aspartic acid units, glutamic acid units, or both, and optionally lysine units, arginine units, or both.

3. The polymer of claim 2 wherein a plurality of aspartic acid or glutamic acid units are covalently linked to poly(ethylene glycol) chains; the molecular weight of the poly(ethylene glycol) chains is each independently about 500 to about 10,000; and the poly(ethylene glycol) chains each optionally terminate in methoxy, amino, or acetamide groups.

4. The polymer of claim 3 wherein the ratio of aspartic acid or glutamic acid units to polyethylene(glycol) chains is about 1:1 to about 20:1.

5. The polymer of claim 2 comprising poly(lysine) units wherein one or more of the lysine side chain amino groups is optionally protected with trifluoroacetate groups or carbonylbenzyloxy groups.

6. The polymer of claim 2 comprising about 5 to about 100 aspartic acid or glutamic acid units; and about 10 to about 150 lysine or arginine units.

7. A polyamide block polymer comprising a first block and a second block; wherein
the first block comprises amino acid units derived from amino acid units with side chains that can have neutral or negative charges;
the second block comprises amino acid units with side chains that can have positive charges;
wherein at least one side chain of an amino acid of the first block is covalently linked to a poly(ethylene glycol) chain through a hydrazide moiety, and
wherein the hydrazide moiety is linked to the poly(ethylene glycol) chain at the N′ nitrogen of the hydrazide through a hydrazone bond.

8. The polymer of claim 7 wherein the first block comprises aspartic acid or glutamic acid units, and the second block comprises lysine or arginine units.

9. The polymer of claim 8 wherein a plurality of aspartic acid or glutamic acid units are covalently linked to poly(ethylene glycol) chains; the molecular weight of the poly(ethylene glycol) chains is each independently about 500 to about 10,000; and the poly(ethylene glycol) chains each optionally terminate in methoxy, amino, or acetamide groups.

10. The polymer of claim 9 wherein the ratio of aspartic acid or glutamic acid units to polyethylene(glycol) chains is about 1:1 to about 20:1.

11. The polymer of claim 8 comprising poly(lysine) units wherein one or more of the lysine side chain amino groups is optionally protected with trifluoroacetate groups or carbonylbenzyloxy groups.

12. The polyamide block polymer of claim 7 comprising at least one block of poly(aspartic acid) or poly(glutamic acid) units and at least one block of poly(lysine) or poly(arginine) units;
wherein at least one side chain of an aspartic acid unit or a glutamic acid units is covalently linked to a poly(ethylene glycol) chain through a hydrazide moiety, and
wherein the hydrazide moiety is linked to the poly(ethylene glycol) chain at the N′ nitrogen of the hydrazide through a hydrazone bond.

13. The polymer of claim 12 wherein a plurality of aspartic acid or glutamic acid units are covalently linked to poly(ethylene glycol) chains; the molecular weight of the poly(ethylene glycol) chains is each independently about 500 to about 10,000; and the poly(ethylene glycol) chains each optionally terminate in methoxy, amino, or acetamide groups.

14. The polymer of claim 12 wherein the ratio of aspartic acid or glutamic acid units to polyethylene(glycol) chains is about 1:1 to about 20:1.

15. The polymer of claim 12 comprising poly(lysine) units wherein one or more of the lysine side chain amino groups is optionally protected with trifluoroacetate groups or carbonylbenzyloxy groups.

16. A micelle composition comprising the polymer of claim 1 and a therapeutic agent, wherein the polymer substantially encapsulates the therapeutic agent and the poly(ethylene glycol) chains of the polymer align toward the outside surface of the micelle.

17. The micelle composition of claim 16 wherein the therapeutic agent comprises a nucleic acid, a gene, a drug, a prodrug, or a combination thereof, and when the therapeutic agent comprises a nucleic acid, the nucleic acid is optionally a plasmid DNA encoding a gene product or RNAi.

18. The polymer of claim 1 comprising formula I: 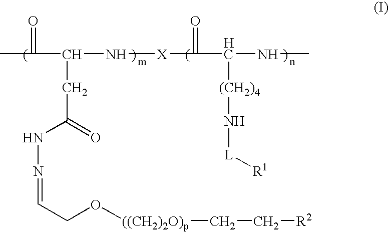
wherein
m is about 5 to about 100;
n is 0, or about 10 to about 150;
p is selected such that the molecular weight of the poly(ethylene glycol) chain is about 500 to about 12,000;
each L is independently a direct bond, carbonyl, or sulfonyl;
each R¹ is independently H, trifluoromethyl, or benzyloxy;
each R² is independently hydrogen, hydroxy, alkyl, alkoxy, —NHR³ wherein R³ is hydrogen, an amino protecting group, or a PEG-capping group; and
X is a direct bond, an organic linking group, or a group of about 1 to about 100 amino acids;
or a salt thereof.

19. The polymer of claim 18 wherein m is about 30 to about 50, n is about 40 to about 60, p is selected such that the molecular weight of the poly(ethylene glycol) chain is about 2,000 to about 10,000; and R² is —NHR³ wherein R³ is an acetyl group.

20. The polymer of claim 1 comprising formula II: 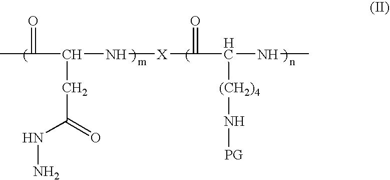
wherein
m is about 5 to about 100; n is 0, or about 10 to about 150; each PG is independently hydrogen or an amino protecting group; and X is a direct bond, an organic linking group, or a group of about 1 to about 100 amino acids;
or a salt thereof.

21. A method of activating a therapeutic prodrug comprising:
contacting the micelle composition of claim 16 and a cell, wherein the micelle composition contains a gene or vector that encodes a therapeutic protein that converts the prodrug to an active drug; and
introducing the prodrug to the vicinity of the cell after the cell has synthesized the therapeutic protein so that the therapeutic protein activates the prodrug to the active drug;
wherein the therapeutic protein is optionally an enzyme.

22. The method of claim 21 wherein amino acid side chain protecting groups have been removed from the polymer of the micelle composition prior to forming the micelle that encapsulates the therapeutic agent.

23. A method of providing RNAi to a cell comprising:
contacting the micelle composition of claim 16 and a cell, wherein the micelle composition contains a gene or vector that encodes RNAi that silences or reduces the expression of a gene in the cell;
wherein the gene or vector is incorporated into the cell and the cell then synthesizes the RNAi thereby silencing or reducing the expression of a gene.

24. A method of delivering a gene, vector, RNAi, or plasmid that encodes a gene product or RNAi, to a cell comprising encapsulating the gene or vector in a micelle comprising the polymer of claim 1; and contacting a cell with the micelle.
